(19) United States
(12) Patent Application Publication
(10) Pub. No.: US 2010/0324152 A1
(43) Pub. Date: Dec. 23, 2010

(54) USE OF 1,2-DECANEDIOL FOR SEBUM REDUCTION AND COSMETIC AND/OR DERMATOLOGICAL FORMULATIONS COMPRISING 1,2-DECANEDIOL

(75) Inventors: Gerhard Schmaus, Hoxter-Bosseborn (DE); Sabine Lange, Holzminden (DE); Joachim Roding, Badenweiler (DE)

Correspondence Address: CONNOLLY BOVE LODGE & HUTZ LLP, 1875 EYE STREET, N.W., SUITE 1100, WASHINGTON, DC, 20006, US

(73) Assignee: SMYRISE GmbH & Co. KG, Holzminden (DE)

(21) Appl. No.: 12/446,190

(22) PCT Filed: Oct. 12, 2007
(86) PCT No.: PCT/EP07/60903

§ 371 Date: May 28, 2010

Related U.S. Application Data
(60) Provisional application No. 60/852,975, filed on Oct. 20, 2006.

(30) Foreign Application Priority Data
Oct. 20, 2006 (EP) 06122665.0

(51) Int. Cl. A61K 31/045; C07C 31/18

(52) U.S. Cl. 514/738; 568/852

(57) ABSTRACT

The present invention relates to a use of 1,2-decanediol for reduction of the sebum concentration of the skin, a cosmetic and/or dermatological formulation for topical application comprising 1,2-decanediol, a method for reduction of the sebum concentration of the skin and a use of 1,2-decanediol and a method for assisting the penetration of one, two, three, four or more active compounds into areas of the skin with increased sebum production.

### CROSS-REFERENCE TO RELATED APPLICATIONS

This application claims benefit of priority to PCT/EP2007/060903, filed on Oct. 12, 2007, which asserts priority to EP 06 1255 665.0 filed on Oct. 20, 2006, and to U.S. Provisional Application No. 60/852,975, filed on Oct. 20, 2006, which are incorporated herein by reference in their entireties.

The present invention relates to a use of 1,2-decanediol for reduction of the sebum concentration of the skin, a cosmetic and/or dermatological formulation for topical application comprising 1,2-decanediol, a method for reduction of the sebum concentration of the skin and a use of 1,2-decanediol and a method for assisting the penetration of one, two, three, four or more active compounds into areas of the skin with increased sebum production.

Seborrhoea means over-production of sebum (sebaceous matter) in a sebaceous gland. Over-production of sebum causes the sebaceous gland follicles to be partly or completely filled with sebum. This sebum often has a very firm consistency and accordingly a low spreading, so that it can be released from the sebaceous gland follicles and can spread over the skin only with great difficulty. The sebum consequently accumulates to an undesirably high degree in the sebaceous glands and forms an ideal nutrient medium for microorganisms, such as, in particular, Propionibacterium acnes, a microorganism involved decisively in the development of acne.

On greasy-oily skin, over-production of sebum leads to an undesirable greasy shine on the skin. In the area of a greasy-oily scalp, over-production of sebum leads to an undesirable greasy shine on the roots of the hair.

On impure skin, over-production of sebum leads to irregularities of the skin, such as e.g. pimples and pustules. Acne is a pathologically changed clinical skin picture of impure skin.

In cases of acne, which usually occurs in puberty, the anaerobic microorganism Propionibacterium acnes (P. acnes) is involved decisively in the development of the condition and decomposes the sebum to glycerin and fatty acids, as a result of which the sebaceous glands are in turn stimulated to produce an increased amount of sebum, these degradation products attacking or destroying the follicle walls. This regularly causes inflammations in the skin (pimples, pustules, nodules, cysts), which often heal only with scarring, as a result of which the visual appearance of the person suffering from impure skin is permanently damaged (W. Umbach [ed.], Kosmetik, Entwicklung, Herstellung and Anwendung kosmetischer Mittel [Cosmetics, Development, Production and Use of Cosmetic Agents], 2nd ed. Thieme Verlag, Stuttgart, 1995).

Conventional products for treatment of greasy and impure skin, such as e.g. aqueous-ethanolic and/or surfactant-containing cleansing products, as a rule have the disadvantage of stressing and drying out the skin and having little care effect. It is a particular disadvantage that the body can react to the use of ethanolic and/or detergent-containing solutions over a relatively long period of time by over-production of sebum, which precisely counteracts the primary therapeutic aim of sebum reduction on greasy-oily, impure skin tending towards acne.

Acne therapeutics are available for topical treatment of acne, such as, for example, potent oxidizing agents, such as e.g. benzoyl peroxide; alpha-hydroxy acids, such as e.g. salicylic acid and lactic acid; aliphatic dicarboxylic acids, such as e.g. azelaic acid; retinoids, such as e.g. tretinoin (synonym: all-trans-retinoic acid), all-trans-retinal and cis-13-retinoic acid (isotretinoin); antiandrogens (5alpha-reductase inhibitors) and antibiotics, such as e.g. clindamycin, tetracycline and erythromycin. An abrasive treatment can moreover be carried out. Corresponding preparations contain aluminium oxide particles or silicone resin.

The activity of the potent oxidizing agents, such as e.g. benzoyl peroxide; the alpha-hydroxy acids, such as e.g. salicylic acid and lactic acid, and the aliphatic dicarboxylic acids, such as e.g. azelaic acid, is attributed here inter alia to inhibition of P. acnes. However, the substances mentioned usually have only a very moderate antimicrobial activity against P. acnes and must therefore be employed in cosmetic and dermatological formulations in a relatively high concentration—benzoyl peroxide e.g. at up to 5 wt. % and azelaic acid at up to 20 wt. %. Due to the high dosage, however, the skin is very highly stressed in an extremely disadvantageous manner, which manifests itself in particular in severe drying out of the skin, which in some cases is often accompanied by severe skin irritations because of the marked reduction in the pH of the skin.

Retinoids have a broad activity spectrum. The action here is based in particular on the normalization of the follicular hyperkeratosis accompanying acne, the disintegration of blackheads and an inhibition of sebum production. A disadvantage, however, is that most retinoids likewise have severely irritating side effects. Isotretinoin moreover shows a teratogenic activity and therefore cannot be employed for treatment of severely greasy, impure skin tending towards acne on pregnant women.

The antibiotics for topical and systemic use, such as e.g. clindamycin, erythromycin and tetracycline, have a potently antimicrobial activity against P. acnes, with MIC values in the region of <1 ppm. In particular, due to the occurrence of resistant P. acnes which is ever more frequently to be observed in clinical practice and the non-specific action spectrum thereof, which leads to severe impairment of the healthy human microflora, the familiar antibiotics of acne therapy are increasingly also viewed with criticism.

For treatment of acne, EP 1 598 064 A1 proposes an active compound combination of alpha- and/or beta-hydroxy acids and alkane-1,2-diols, it being reported that the combination of 1 wt. % lactic acid with 0.2 wt. % 1,2-decanediol has an improved antibacterial activity of the combination with respect to P. acnes compared with the individual constituents, in particular 0.2 wt. % 1,2-decanediol. On administration solely of 0.2 wt. % 1,2-decanediol, there is as good as no antibacterial action to be seen in the suspension test.

An object of the present invention is thus to provide a compound and/or a formulation for reduction of the sebum concentration in the skin in order to reduce and/or prevent the greasy shine in particular on greasy-oily skin and/or to achieve on impure skin, which is distinguished in particular by skin irregularities, such as e.g. pimples and pustules, a reduction in the skin irregularities.

In addition, the compound and/or the formulation should have a care effect and moreover should not stress and/or dry out the skin and/or hair.

The above objects are achieved completely or at least in part by the subject matter of the present invention as reproduced in particular in the claims.

One embodiment of the present invention relates to a use of 1,2-decanediol for reduction of the sebum concentration of the skin.

A further embodiment of the present invention relates to a use of 1,2-decanediol for the preparation of a cosmetic and/or dermatological composition for topical application for reduction of the sebum concentration of the skin.

A further embodiment of the present invention relates to a cosmetic and/or dermatological formulation for topical application, characterized in that the formulation comprises the following constituents or consists of these constituents:
an amount of 1,2-decanediol which is sufficient to reduce the sebum concentration of the skin,
optionally one, two, three, four, five, six, seven, eight, nine, ten or more cleansing auxiliary substances,
one, two, three, four or more active compounds chosen from the group consisting of benzoyl peroxide; salicylic acid; lactic acid; azelaic acid; retinoids, preferably all-trans-retinoic acid, all-trans-retinal, cis-13-retinoic acid and adaptalene; antibiotics, preferably clindamycin, erythromycin and tetracycline; sulfur; chlorhexidine; triclosan; farnesol; phenoxyethanol; isoflavonoids, preferably genistein, daidzein, genistin and daizin, and antiandrogens, preferably 5alpha-reductase inhibitors and
optionally one or more further auxiliary substances and/or additives with the proviso that
if the formulation contains 1 wt. % of 1,2-decanediol, then the formulation may not at the same time contain 1 wt. % lactic acid or 2 wt. % lactic acid,
if the formulation contains 0.5 wt. % 1,2-decanediol, then the formulation may not at the same time contain 0.5 wt. % salicylic acid or 1 wt. % salicylic acid or 0.5 wt. % lactic acid and 1 wt. % salicylic acid,
if the formulation contains 0.4 wt. % 1,2-decanediol, then the formulation may not at the same time contain 1 wt. % lactic acid and 0,5 wt. % salicylic acid,
if the formulation contains 0.3 wt. % 1,2-decanediol, then the formulation may not at the same time contain 2.5 wt. % lactic acid or 0,2 wt. % salicylic acid and 1 wt. % lactic acid,
if the formulation contains 0.2 wt. % 1,2-decanediol, then the formulation may not at the same time contain 2 wt. % lactic acid or 1 wt. % lactic acid or 0.5 wt. % lactic acid, and
if the formulation contains 0.1 wt. % 1,2-decanediol, then the formulation may not at the same time contain 1 wt. % lactic acid.

A further embodiment of the present invention relates to a method for reduction of the sebum concentration of the skin, characterized in that the method comprises the following steps or consists of the following steps:
a) provision of 1,2-decanediol;
b) preparation of a formulation according to the invention, as described above, and
c) application of the formulation prepared according to b) to greasy-oily and/or impure skin, the amount of 1,2-decanediol being sufficient to reduce the sebum concentration of the skin.

A further embodiment of the present invention relates to a use of 1,2-decanediol to assist the penetration of one, two, three or more active compounds chosen from the group consisting of benzoyl peroxide, salicylic acid, lactic acid, azelaic acid, retinoids, antibiotics, sulfur, chlorhexidine, triclosan, farnesol, phenoxyethanol, isoflavonoids and antiandrogens in areas of the skin with increased sebum production.

A further embodiment of the present invention relates to a method for assisting the penetration of one, two, three or more active compounds chosen from the group consisting of benzoyl peroxide, salicylic acid, lactic acid, azelaic acid, retinoids, antibiotics, sulfur, chlorhexidine, triclosan, farnesol, phenoxyethanol, isoflavonoids and antiandrogens in areas of the skin with increased sebum production, characterized in that the method comprises the following steps or consists of the following steps:
a) provision of 1,2-decanediol,
b) provision of one, two, three or more active compounds chosen from the group consisting of benzoyl peroxide, salicylic acid, lactic acid, azelaic acid, retinoids, antibiotics, sulfur, chlorhexidine, triclosan, farnesol, phenoxyethanol, isoflavonoids and antiandrogens,
c) preparation of a cosmetic and/or dermatological formulation for topical application comprising (or consisting of)
   an amount of 1,2-decanediol which is sufficient to reduce the sebum concentration of the skin,
   an active amount of one, two, three, four or more active compounds chosen from the group consisting of benzoyl peroxide, salicylic acid, lactic acid, azelaic acid, retinoids, antibiotics, sulfur, chlorhexidine, triclosan, farnesol, phenoxyethanol, isoflavonoids and antiandrogens
   optionally one, two, three, four, five, six, seven, eight, nine, ten or more cleansing auxiliary substances and
   optionally one or more further auxiliary substances and/or additives with the proviso that
   if the formulation contains 1 wt. % of 1,2-decanediol, then the formulation may at the same time not contain 1 wt. % lactic acid or 2 wt. % lactic acid,
   if the formulation contains 0.5 wt. % 1,2-decanediol, then the formulation may at the same time not contain 0.5 wt. % salicylic acid or 1 wt. % salicylic acid or 0.5 wt. % lactic acid and 1 wt. % salicylic acid,
   if the formulation contains 0.4 wt. % 1,2-decanediol, then the formulation may at the same time not contain 1 wt. % lactic acid and 0.5 wt. % salicylic acid,
   if the formulation contains 0.3 wt. % 1,2-decanediol, then the formulation may at the same time not contain 2.5 wt. % lactic acid or 0.2 wt. % salicylic acid and 1 wt. % lactic acid,
   if the formulation contains 0.2 wt. % 1,2-decanediol, then the formulation may at the same time not contain 2 wt. % lactic acid or 1 wt. % lactic acid or 0.5 wt. % lactic acid, and
   if the formulation contains 0.1 wt. % 1,2-decanediol, then the formulation may at the same time not contain 1 wt. % lactic acid, and
d) application of the formulation prepared according to c) to relevant areas of the skin.

It is to be pointed out that in the context of the present embodiments according to the invention, the term 1,2-decanediol includes both (a) the enantiomer in the 2S configuration and (b) the enantiomer in the 2R configuration as well as (c) any desired mixtures of decanediols in the 2S and 2R configuration. For commercial reasons, it is indeed particularly advantageous to employ the racemate of 1,2-decanediol for treatment of greasy-oily and impure skin and for prophylaxis and/or treatment of seborrhoea, in particular acne, since these are particularly readliy accessible by synthesis, but the pure enantiomers or non-racemic mixtures of these enantiomers are likewise suitable for the purposes according to the invention.

The above embodiments of the present invention are based on the finding that 1,2-decanediol significantly reduces the dropping point and therefore the fluidity of sebum. This lowering of the dropping point of sebum has been demonstrated with the aid of an in vitro study on a model sebum (see Example 1). This specific property of 1,2-decanediol means that the spreading ability of the sebum is increased by the increased fluidity of the sebum, and the sebum thus has an increased ability to spread out from the sebaceous gland follicles on to the skin.

With the aid of clinical in vivo studies (Example 2), it has been shown that by using 1,2-decanediol it was possible for the sebum concentration of the skin of adolescent test subjects aged 17-19 years with greasy-oily and/or impure skin, in particular tending towards acne, to be reduced significantly.

In the context of the present invention, the term “skin” means human or animal skin.

In the present invention, “reduction of the sebum concentration of the skin” is to be understood as meaning a significant reduction of the sebum concentration of the skin, including a reduction of the sebum concentration in the skin, in particular in the sebaceous gland follicles, and/or on the skin and/or, on the hair-covered scalp, on the hair. This reduction of the sebum concentration of the skin can be determined, for example, by means of sebumetry, in particular by means of a Sebumeter®SM 810, obtainable from Courage & Khazaka, Köln, Germany.

The cause of this has not yet been clarified completely, but is certainly to be attributed to an improved solubility of the sebum caused by fluidization and an associated improved mechanical removal of the sebum. On the basis of the amphiphilic, solubilizing character of 1,2-decanediol, surfactant-like effects of 1,2-decanediol can also contribute here to the improved removal of the highly lipophilic sebum.

The use of 1,2-decanediol consequently leads to a reduction in the sebum concentration of the skin, as a result of which greasy shine on greasy-oily skin and/or skin irregularities on impure skin can be reduced.

It is furthermore assumed that the reduction in the sebum concentration of the skin, in particular in the sebaceous gland follicles, but also on the surface of the skin, assists the penetration of one, two, three, four or more active compounds chosen from the group consisting of benzoyl peroxide; salicylic acid; lactic acid; azelaic acid; retinoids, preferably all-trans-retinoic acid, all-trans-retinal, cis-13-retinoic acid and adaptalene; antibiotics, preferably clindamycin, erythromycin and tetracycline; sulfur; chlorhexidine; triclosan; farnesol; phenoxyethanol; isoflavonoids, preferably genistein, daidzein, genistin and daizin, and antiandrogens, preferably 5a-reductase inhibitors, in areas of the skin with increased sebum production.

Preferably one, two, three or more active compounds are chosen from the group consisting of benzoyl peroxide, salicylic acid, lactic acid, azelaic acid, all-trans retinoic acid, all-trans retinal, cis-13-retinoic acid, adaptalen, clindamycin, erythromycin, tetracycline, sulfur, chlorhexidine, triclosan, farnesol, phenoxyethanol, genistein, daidzein, genistin, daizin and 5α-reductase inhibitor.

More preferably one, two, three or more active compounds are chosen from the group consisiting of benzoyl peroxide, azelaic acid, all-trans retinoic acid, all-trans retinal, cis-13-retinoic acid, adaptalen, clindamycin, erythromycin, tetracycline, sulfur, chlorhexidine, triclosan, farnesol, phenoxyethanol, genistein, daidzein, genistin, daizin and 5α-reductase inhibitor.

Even more preferred are one, two, three or more active compounds chosen from the group consisting of benzoyl peroxide, azelaic acid, all-trans-retinoic acid, all-trans-retinal, cis-13-retinoic acid, adaptalene, clindamycin, erythromycin and tetracycline.

Even more preferred are one, two, three or more active compounds chosen from the group consisting of benzoyl peroxide, azelaic acid, adaptalene, clindamycin, erytrhomacin and tetracycline.

In a further preferred embodiment all above mentioned cosmetic and/or dermatological formulations for topical application do not comprise an eucalyptus extract and a ginger root extract; and/or a Thujopsis dolabrata extract and a focus vesiculosus extract; and/or a Phellodendron Amurense extract, olive oil and lavender oil; and/or a chamomile extract.

It is assumed that by the reduction in the sebum, the surface of the skin is available to the active compound or the particular active compounds for the particular action to a greater extent.

Areas of the skin with increased sebum production are distinguished by an increased number of and/or activity of the sebaceous gland follicles and are preferentially to be found in the area of the face, and more preferentially on the forehead, nose, cheek, chin, hair-covered scalp; front and rear sweat channel; neck; chest and back. The areas of the forehead, nose, cheek, chin, hair-covered scalp, neck, chest and back are more preferential.

The antimicrobial action of aliphatic alkane-1,2-diols is already adequately known per se. Nevertheless, none of the documents of the prior art reports that 1,2-decanediol causes a reduction of the sebum concentration in the skin.

WO 03/000220 (Dragoco Gerberding & Co. KG) describes the use of 1,2-decanediol merely against bacteria which cause body odour. A reduction of the sebum concentration of the skin by using 1,2-decanediol is not disclosed there.

A number of further documents are either concerned with 1,2-decanediol without thereby disclosing the reduction of the sebum concentration of the skin by using 1,2-decanediol, or concerned with the antimicrobial action of other 1,2-alkanediols without thereby describing 1,2-decanediol or the property the reduction of the sebum concentration of the skin. Reference may also be made in this respect to the following documents: FR 2,755,371; JP 11 310506; WO 99/11237; WO 99/56715; WO 99/56716; JP 20 0044419; JP 11 335258; JP 10 053510; J. Food Sc. 42(3), 699-701; EP 1 000 542; DE 199 24 496 and JP 20 0148720.

U.S. Pat. No. 6,123,953 states that unbranched 1,2-alkanediols having a chain length of 5 to 14 C atoms in total and preferably 6 to 8 C atoms are suitable as agents having antimicrobial activity for topical application to the skin, and here in particular for treatment of acne, for treatment of capillary layers, for disinfection of small wounds (scratches), for disinfection of the hands of surgeons and patients and for disinfection of the udders of milk-yielding animals. 1,2-Decanediol is not mentioned explicitly. U.S. Pat. No. 6,123,953 furthermore discloses that formulations which comprise a gel of the glyceryl polymethacrylate type, in addition to a 1,2-alkanediol, can be employed in a low concentration for treatment of acne, skin complaints, impetigo, microorganism-based body odours, athlete's foot and the like. This usability is attributed here to a synergistic interaction between the gel and the alkanediol. 1,2-Octanediol is stated as the preferred 1,2-alkanediol for treatment of acne in this specification. A reduction in the sebum concentration in the skin by these 1,2-alkanediols is not disclosed there.

As already described above, EP 1 598 064 A1 discloses the use of 1,2-decanediol in combination with alpha- and/or beta-hydroxy acids in anti-acne products, only the synergistic interaction of this combination in respect of the antimicrobial property on P. acnes being dealt with here. Nevertheless, a reduction in the sebum concentration of the skin is not disclosed in EP 1 598 064 A1.

Summarizing, this means that the prior art is merely based on the fact that 1,2-alkanediols, in particular 1,2-decanediol, have antibacterial properties with respect to Propionibacterium acnes and to bacteria which cause body odour. Accordingly, there is no teaching that 1,2-decanediol causes a reduction of the sebum concentration of the skin.

The preferred embodiments of the present invention are presented in the dependent claims and described in more detail in the following.

Compositions for reduction of the sebum concentration of the skin are preferably chosen from the product groups consisting of:

cleansing emulsion, cleansing milk, cream-like washing emulsion, cleansing washing gel, clarifying and/or care facial water, cleansing cloth and shampoo and/or hair treatment rinse in the scalp area. Frequent auxiliary substances and/or additives which are preferably contained in such products and are particularly suitable, preferably one, two, three, four, five, six, seven eight, nine, ten or more, more preferably three or more, still more preferably four or more auxiliary substances and/or additives, are chosen from the group consisting of:

water; ethanol; surfactants, preferably sodium lauryl sulfate, lauryl glucoside and cocamidopropylbetaine; polyethylene glycol derivatives, preferably for example PEG-8 and PEG-20 (PEG corresponds to polyethylene glycol); polyethylene glycol esters; preferably PEG-5 ethylhexanoate; skin moisture-regulating substances, preferably glycerin, propylene glycol and butylene glycol, urea and sorbitol; readily spreading cosmetic esters, preferably ethylhexyl ethylhexanoate, ethylhexyl isononanoate and cetearyl ethylhexanoate; and in specific uses optionally skin soothing and/or antiinflammatory additives, preferably alpha-bisabolol, panthenol, allantoin, anthranilic acid amides—in particular those mentioned in WO 2004/047833, preferably dihydroavenanthramide D—and plant extracts, preferably witch hazel extracts, ginger extracts and oat extracts; talc and bentonite.

The cosmetic and/or dermatological composition for topical application for reduction of the sebum concentration of the skin is preferably employed as a composition for care, prophylaxis and/or treatment of seborrhoea.

The abovementioned product groups and the preferred auxiliary substances and additives for compositions for reduction of the sebum concentration of the skin are also preferred as compositions for care, prophylaxis and/or treatment of seborrhoea.

A composition for care, prophylaxis and/or treatment of seborrhoea is furthermore preferably employed for care, prophylaxis and/or treatment of greasy-oily and/or impure skin.

The abovementioned product groups and the preferred auxiliary substances and additives for compositions for reduction of the sebum concentration of the skin are also preferred for care, prophylaxis and/or treatment of greasy-oily and/or impure skin.

Since the seborrhoeic condition of the skin is an ideal nutrient medium for bacterial and fungal growth and consequently for the development of acne, a composition for prophylaxis and/or treatment of impure skin is likewise a preferred composition for prophylaxis and/or treatment of acne.

The abovementioned product groups and the preferred auxiliary substances and additives for compositions for reduction of the sebum concentration of the skin are also preferred as compositions for prophylaxis and/or treatment of acne.

A preferred cosmetic formulation for topical application comprises the following constituents or consists of the following constituents:
an amount of 1,2-decanediol which is sufficient to reduce the sebum concentration of the skin,
one, two, three, four, five, six, seven, eight, nine, ten or more, preferably three or more, more preferably four or more cleansing auxiliary substances,
optionally one or more further auxiliary substances and/or additives.

Such a cosmetic formulation is particularly suitable for cleansing greasy-oily and/or impure skin.

Preferably, one, two, three, four, five, six, seven, eight, nine, ten or more, preferably three or more, more preferably four or more cleansing auxiliary substances are chosen from the group consisting of:

water; ethanol; surfactants, preferably sodium lauryl sulfate, lauryl glucoside and cocamidopropylbetaine; polyethylene glycol derivatives, preferably PEG-8 and PEG-20; polyethylene glycol esters, preferably PEG-5 ethylhexanoate; skin moisture-regulating substances, preferably polyols—of these preferably glycerin, propylene glycol or butylene glycol—, urea and sorbitol; readily spreading cosmetic esters, preferably ethylhexyl ethylhexanoate, ethylhexyl isononanoate and cetearyl ethylhexanoate; and in specific uses optionally skin soothing and/or antiinflammatory compounds, preferably alpha-bisabolol, panthenol, allantoin, anthranilic acid derivatives—in particular those mentioned in WO 2004/047833, of these preferably dihydroavenanthramide D—and plant extracts—of these preferably witch hazel extracts, ginger extracts and oat extracts, and talc and bentonite.

A further preferred cosmetic and/or dermatological formulation for topical application comprises the following constituents or consists of the following constituents:
an amount of 1,2-decanediol which is sufficient to reduce the sebum concentration of the skin,
one, two, three, four or more active compounds chosen from the group consisting of benzoyl peroxide; salicylic acid; lactic acid; azelaic acid; retinoids, preferably all-trans-retinoic acid, all-trans-retinal, cis-13-retinoic acid and adaptalene; antibiotics, preferably clindamycin, erythromycin and tetracycline; sulfur; chlorhexidine; triclosan; farnesol; phenoxyethanol; isoflavonoids, preferably genistein, daidzein, genistin and daizin, and antiandrogens, preferably 5alpha-reductase inhibitors and
optionally one or more further auxiliary substances and/or additives
   with the proviso
if the formulation contains 1 wt. % of 1,2-decanediol, then the formulation may at the same time not contain 1 wt. % lactic acid or 2 wt. % lactic acid,
if the formulation contains 0.5 wt. % 1,2-decanediol, then the formulation may at the same time not contain 0.5 wt. % salicylic acid or 1 wt. % salicylic acid or 0.5 wt. % lactic acid and 1 wt. % salicylic acid,
if the formulation contains 0.4 wt. % 1,2-decanediol, then the formulation may at the same time not contain 1 wt. % lactic acid and 0.5 wt. % salicylic acid,
if the formulation contains 0.3 wt. % 1,2-decanediol, then the formulation may at the same time not contain 2.5 wt. % lactic acid or 0.2 wt. % salicylic acid and 1 wt. % lactic acid,
if the formulation contains 0.2 wt. % 1,2-decanediol, then the formulation may at the same time not contain 2 wt. % lactic acid or 1 wt. % lactic acid or 0.5 wt. % lactic acid, and
if the formulation contains 0.1 wt. % 1,2-decanediol, then the formulation may at the same time not contain 1 wt. % lactic acid.

In a further preferred embodiment of the cosmetic and/or dermatological formulation for topical application, one, two, three or more active compounds are chosen from the group consisting of benzoyl peroxide, salicylic acid, azelaic acid, all-trans-retinoic acid, all-trans-retinal, cis-13-retinoic acid, adaptalene, clindamycin, erythromycin and tetracycline.

Preferably one, two, three or more active compounds are chosen from the group consisting of benzoyl peroxide, salicylic acid, lactic acid, azelaic acid, all-trans retinoic acid, all-trans retinal, cis-13-retinoic acid, adaptalen, clindamycin, erythromycin, tetracycline, sulfur, chlorhexidine, triclosan, farnesol, phenoxyethanol, genistein, daidzein, genistin, daizin and 5a-reductase inhibitor.

More preferably one, two, three or more active compounds are chosen from the group consisting of benzoyl peroxide, azelaic acid, all-trans retinoic acid, all-trans retinal, cis-13-retinoic acid, adaptalen, clindamycin, erythromycin, tetracycline, sulfur, chlorhexidine, triclosan, farnesol, phenoxyethanol, genistein, daidzein, genistin, daizin and 5a-reductase inhibitor.

Even more preferred are one, two, three or more active compounds chosen from the group consisting of benzoyl peroxide, azelaic acid, all-trans-retinoic acid, all-trans-retinal, cis-13-retinoic acid, adaptalene, clindamycin, erythromycin and tetracycline.

Even more preferred are one, two, three or more active compounds chosen from the group consisting of benzoyl peroxide, azelaic acid, adaptalene, clindamycin, erytrhomacin and tetracycline.

In a further preferred embodiment all above mentioned cosmetic and/or dermatological formulations for topical application do not comprise an eucalypt extract and a ginger root extract; and/or a Thujopsis dolabrata extract and a focus vesiculosus extract; and/or a Phellodendron Amurense extract, olive oil and lavender oil; and/or a chamomile extract.

In particular not according to the present invention is a cosmetic or pharmaceutical formulation for topical application as set out before, which is an aqueous solution containing 1% by weight of decane-1,2-diol, 3% by weight of glycerine, 2% by weight of polysorbate (Tween®20) and water as an excipient qsp 100%.

The amount of one, two, three or more (preferred) active compounds in the formulations according to the invention is in each case preferably 0.0001 to 20 wt. %, particularly preferably 0.0001-10 wt. %, in particular 0.001-5 wt. %, in each case based on the total weight of the formulation, the total amount of all (preferred) active compounds in the formulations according to the invention being 0.0001 to 20 wt. %, preferably 0.001 to 10 wt. %.

Since acne is usually accompanied by inflammatory processes, the cosmetic and/or dermatological formulations according to the invention which comprise 1,2-decanediol can particularly advantageously also comprise one, two, three or more, preferably one, two or three antiinflammatory and/or redness- and/or further itching-alleviating active compounds (antiinflammatories). All the antiinflammatory or redness- and/or itching-alleviating active compounds which are suitable or usual for cosmetic and/or dermatological uses can be used here.

The antiinflammatory and redness- and/or itching-alleviating active compounds are advantageously chosen from the group consisting of steroidal antiinflammatory active compounds of the corticosteroid type, particularly preferably hydrocortisone; hydrocortisone derivatives, preferably hydrocortisone 17-butyrate, dexamethasone, dexamethasone phosphate, methylprednisolone or cortisone, it being possible for the list to be extended by addition of further steroidal antiinflammatories.

Non-steroidal antiinflammatory and redness- and/or itching-alleviating active compounds can also preferably be employed alternatively or cumulatively. There are to be mentioned here by way of example oxicams, preferably piroxicam or tenoxicam; salicylates, preferably aspirin, Disalcid, Solprin or fendosal; acetic acid derivatives, preferably diclofenac, fenclofenac, indomethacin, sulindac, tolmetin or clindanac; fenamates, preferably mefenamic, meclofenamic, flufenamic or niflumic; propionic acid derivatives, preferably ibuprofen, naproxen or benoxaprofen, or pyrazoles, preferably phenylbutazone, oxyphenylbutazone, febrazone or azapropazone.

Antiinflammatory and redness- and/or itching-alleviating active compounds which occur naturally can also preferably be employed alternatively or cumulatively. Suitable substances occur in plant extracts, specifically highly active plant extract fractions and highly pure active substances isolated from plant extracts, and are preferably chosen from camomile, aloe vera, Commiphora species, Rubia species, willow, rose-bay willow herb, oats, calendula, arnica, St. John's wort, honeysuckle, rosemary, Melissa, ginger, Passiflora incarnate, witch hazel, Pueraria, Dianthus or Echinacea, as well as pure substances, preferably, inter alia, bisabolol, apigenin, apigenin 7-glucoside, rosemary acid, boswellic acid, phytosterols, glycyrrhizic acid, glabridin, licochalcone A, gingerols and anthranilic acid amides, preferably, in particular, avenanthramide or dianthramide.

The formulations comprising 1,2-decanediol can thus also comprise mixtures of two or more, preferably two or three antiinflammatory or redness- and/or itching-alleviating active compounds from the group consisting of steroidal antiinflammatory compounds, non-steroidal antiinflammatories and/or natural occurring antiinflammatories.

The amount of antiinflammatory or redness- and/or itching-alleviating active compounds (one or more compounds) in the formulations according to the invention is preferably 0.0001 to 20 wt. %, particularly preferably 0.0001-10 wt. %, more preferably 0.001-5 wt. %, in each case based on the total weight of the formulation.

Preferably, the abovementioned cosmetic and/or dermatological formulations according to the invention for topical application comprise one, two, three, four, five, six, seven, eight, nine, ten or more, preferably three or more, more preferably four or more cleansing auxiliary substances chosen from the group consisting of:

water; ethanol; surfactants, preferably sodium lauryl sulfate, lauryl glucoside and cocamidopropylbetaine; polyethylene glycol derivatives, preferably PEG-8 and PEG-20; polyethylene glycol esters, preferably PEG-5 ethylhexanoate; skin moisture-regulating substances, preferably polyols—of these preferably glycerol, propylene glycol or butylene glycol, urea and sorbitol; readily spreading cosmetic esters, preferably ethylhexyl ethylhexanoate, ethylhexyl isononanoate and cetearyl ethylhexanoate; and in specific uses optionally skin soothing and/or antiinflammatory compounds, preferably alpha-bisabolol, panthenol, allantoin, anthranilic acid derivatives—in particular those mentioned in WO 2004/047833, of these preferably dihydroavenanthramide D—and plant extracts, and talc and bentonite.

1,2-Decanediol is employed in the present cosmetic and/or dermatological formulations according to the invention for topical application in a content of from 0.1 to 10 wt. % and preferably in a concentration of from 0.2 to 5 wt. % and very particularly preferably in a concentration of from 0.5 to 2 wt. %, in each case based on the total weight of the formulation.

In this context, 1,2-decanediol can be employed in the most diverse forms in the present cosmetic and/or dermatological formulations according to the invention for topical application. They can thus be e.g. a solution (e.g. aqueous, aqueous-alcoholic or alcoholic solution, alcoholic here not meaning ethanolic), an emulsion of the water-in-oil (W/O) type or of the oil-in-water (O/W) type, or a multiple emulsion, for example of the water-in-oil-in-water (W/O/W) or oil-in-water-in-oil (O/W/O) type (in each case also in the form of silicone oil emulsions), a hydrodispersion or lipodispersion, a Pickering emulsion, a solid stick or also an aerosol. The impregnation of a water-insoluble substrate with the 1,2-decanediol according to the invention (e.g. a cloth, a nonwoven, a pad) may also be advantageous, it being possible for this application form to be either a dry-feeling or a moist-feeling substrate. Further advantageous presentation forms of the 1,2-decanediol are creams, ointments, hydrodispersions, lotions, tinctures, pump sprays, aerosol sprays, aqueous solutions, cleansing substrates and the like. The aqueous phase in this context can also contain, in addition to water, other constituents, for example diols or polyols of low C number and ethers thereof, preferably propylene glycol, glycerin, ethylene glycol, ethylene glycol monoethyl or monobutyl ether, propylene glycol monomethyl, monoethyl or monobutyl ether, diethylene glycol monomethyl or monoethyl ether and analogous products, and furthermore alcohols of low C number, e.g. isopropanol, 1,2-propanediol and glycerin, and, in particular, one or more thickening agents, which can advantageously be chosen from the group consisting of silicon dioxide, aluminium silicates, polysaccharides and derivatives thereof, e.g. hyaluronic acid, xanthan gum and hydroxypropylmethylcellulose, particularly advantageously from the group consisting of polyacrylates, preferably a polyacrylate from the group consisting of the so-called Carbopols, for example Carbopols of the types 980, 981, 1382, 2984 and 5984, in each case individually or in combination.

Preferred cosmetic and/or dermatological formulations for topical application can furthermore be chosen from the following product groups:

cleansing emulsion, cleansing milk, cream-like washing emulsion, cleansing washing gel, clarifying and/or care facial water, cleansing cloth and shampoo and/or hair treatment rinse in the scalp area.

1,2-Decanediol can be combined with a large number of further auxiliary substances and/or additives, 1,2-decanediol leading at least in some cases to assisting the penetration of auxiliary substances and/or additives which have an action in areas of the skin with increased sebum production, in particular in sebaceous gland follicles, into these areas. Preferred cosmetic and/or dermatological formulations and products according to the invention thereby result:

Combination with Skin Moisture-Regulating Compounds:

1,2-Decanediol can particularly advantageously be additionally combined with one, two, three or more skin moisture-regulating products. Cosmetic formulations which comprise 1,2-decanediol can therefore advantageously additionally comprise the following moisture retention regulators: sodium lactate, urea, urea derivatives, glycerin, diols, such as propylene glycol, hexylene glycol, 1,2-pentanediol, 1,2-hexanediol, 1,2-octanediol or also mixtures of the diols mentioned, such as, in particular, mixtures of 1,2-hexanediol and 1,2-octanediol, collagen, elastin or hyaluronic acid, diacyl adipates, petrolatum, urocanic acid, lecithin, panthenol, phytantriol, lycopene, (pseudo-)ceramides, glycosphingolipids, cholesterol, phytosterols, chitosan, chondroitin sulfate, lanolin, lanolin esters, amino acids, alpha-hydroxy acids (e.g. citric acid, lactic acid, malic acid) and derivatives thereof, mono-, di- and oligosaccharides, such as, for example, glucose, galactose, fructose, mannose, laevulose and lactose, polysugars, such as β-glucans, in particular 1,3-1,4-β-glucan from oats, alpha-hydroxy-fatty acids, triterpenic acids, such as betulic acid or ursolic acid, and algae extracts.

The use concentration of the moisture retention regulators is, depending on the substance, in the concentration range of from 0.1 to 10 wt. % and preferably in the concentration range of 0.5-5 wt. %, in each case based on the total weight of the ready-to-use cosmetic and/or dermatological end product. These data apply in particular to diols which are advantageously to be used, such as hexylene glycol, 1,2-pentanediol, 1,2-hexanediol and 1,2-octanediol, and mixtures of 1,2-hexanediol and 1,2-octanediol.

Combination with Cooling Active Compounds:

1,2-Decanediol can particularly advantageously be additionally combined with one, two, three or more cooling active compounds. Individual cooling active compounds which are preferred for use in the context of the present invention are listed in the following. The person skilled in the art can supplement the following list with a large number of further cooling active compounds; the cooling active compounds listed can also be employed in combination with one another: l-menthol, d-menthol, racemic menthol, menthone glycerin acetal (trade name: FrescolaCMGA), menthyl lactate (trade name: Frescolat®ML, menthyl lactate is preferably l-menthyl lactate, in particular l-menthyl l-lactate), substituted menthyl-3-carboxylic acid amides (e.g. menthyl-3-carboxylic acid N-ethylamide), 2-isopropyl-N-2,3-trimethylbutanamide, substituted cyclohexanecarboxylic acid amides, 3-menthoxypropane-1,2-diol, 2-hydroxyethyl menthyl carbonate, 2-hydroxypropyl menthyl carbonate, N-acetylglycine menthyl ester, isopulegol, menthyl hydroxycarboxylic acid esters (e.g. menthyl 3-hydroxybutyrate), monomenthyl succinate, 2-mercaptocyclodecanone, menthyl 2-pyrrolidin-5-onecarboxylate, 2,3-dihydroxy-p-menthane, 3,3,5-trimethylcyclohexanone glycerin ketal, 3-menthyl 3,6-di- and -trioxaalkanoates, 3-menthyl methoxyacetate and icilin.

Preferred cooling active compounds are: l-menthol, d-menthol, racemic menthol, menthone glycerin acetal (trade name: Frescolat®MGA), menthyl lactate (preferably l-menthyl lactate, in particular l-menthyl l-lactate, trade name: Frescolat®ML), substituted menthyl-3-carboxylic acid amides (e.g. menthyl-3-carboxylic acid N-ethylamide), 2-isopropyl-N-2,3-trimethylbutanamide, substituted cyclohexanecarboxylic acid amides, 3-menthoxypropane-1,2-diol, 2-hydroxyethyl menthyl carbonate, 2-hydroxypropyl menthyl carbonate and isopulegol.

Particularly preferred cooling active compounds are: l-menthol, racemic menthol, menthone glycerin acetal (trade name: FrescolaCMGA), menthyl lactate (preferably l-menthyl lactate, in particular l-menthyl l-lactate, trade name: Frescolat®ML), 3-menthoxypropane-1,2-diol, 2-hydroxyethyl menthyl carbonate and 2-hydroxypropyl menthyl carbonate.

Very particularly preferred cooling active compounds are: l-menthol, menthone glycerin acetal (trade name: Frescolat®MGA) and menthyl lactate (preferably l-menthyl lactate, in particular l-menthyl l-lactate, trade name: Frescolat®ML).

The use concentration of the cooling active compounds to be employed is, depending on the substance, preferably in the concentration range of from 0.01 to 20 per cent by weight and preferably in the concentration range of from 0.1 to 5 per cent by weight, in each case based on the total weight of a ready-to-use cosmetic or pharmaceutical end product.

Combination with Osmolytes:

1,2-Decanediol can furthermore be employed together with one, two, three or more osmolytes. Osmolytes which may be mentioned by way of example are: substances from the group consisting of sugar alcohols (myo-inositol, mannitol, sorbitol), quaternary amines, such as taurine, choline, betaine, betaine-glycine and ectoin, diglycerin phosphate, phosphorylcholine, glycerophosphorylcholines, amino acids, preferably glutamine, glycine, alanine, glutamate, aspartate or proline, phosphatidylcholine, phosphatidylinositol and inorganic phosphates, as well as polymers of the compounds mentioned, preferably proteins, peptides, poly-amino acids and polyols. These osmolytes at the same time have a skin moisturizing action.

Combination with Care Substances:

Care substances which can be combined with 1,2-decanediol include animal and/or plant fats and oils, such as olive oil, sunflower oil, refined soya oil, palm oil, sesame oil, rapeseed oil, almond oil, borage oil, evening primrose oil, coconut oil, shea butter, jojoba oil, sperm oil, beef tallow, neat's foot oil and lard, and optionally further care constituents, such as, for example, fatty alcohols having 8-30 C atoms. The fatty alcohols used here can be saturated or unsaturated and linear or branched.

Care substances which can be particularly preferably combined with 1,2-decanediol moreover also include, in particular,
ceramides, where ceramides are understood as meaning N-acylsphingosins (fatty acid amides of sphingosin) or synthetic analogues of such lipids (so-called pseudo-ceramides), which significantly improve the water retention capacity of the stratum corneum.
phospholipids, for example soya lecithin, egg lecithin and cephalins
vaseline, paraffin oils and silicone oils; the latter include, inter alia, dialkyl- and alkylarylsiloxanes, such as dimethylpolysiloxane and methylphenylpolysiloxane, as well as alkoxylated and quaternized derivatives thereof.

Combination with preservatives or chelating agents:

Cosmetic formulations which comprise 1,2-decanediol can also comprise one, two, three, four or more active compounds for preserving cosmetic products and perspiration-inhibiting active compounds (antiperspirants) and (metal) chelating agents.

Preservatives which are preferably chosen here are those such as benzoic acid and its esters and salts, propionic acid and its esters and salts, salicylic acid and its esters and salts, 2,4-hexadienoic acid (sorbic acid) and its esters and salts, formaldehyde and paraformaldehyde, 2-hydroxybiphenyl ether and its salts, 2-zinc-sulfidopyridine N-oxide, inorganic sulfites and bisulfites, sodium iodate, chlorobutanolum, 4-ethylmercury-(II)5-amino-1,3-bis(2-hydroxybenzoic acid, its salts and esters, dehydracetic acid, formic acid, 1,6-bis(4-amidino-2-bromophenoxy)-n-hexane and its salts, the sodium salt of ethylmercury-(II)-thiosalicylic acid, phenylmercury and its salts, 10-undecylenic acid and its salts, 5-amino-1,3-bis(2-ethylhexyl)-5-methyl-hexahydropyrimidine, 5-bromo-5-nitro-1,3-dioxane, 2-bromo-2-nitro-1,3-propanediol, 2,4-dichlorobenzyl alcohol, N-(4-chlorophenyl)-N′-(3,4-dichlorophenyl)-urea, 4-chloro-m-cresol, 2,4,4′-trichloro-2′-hydroxy-diphenyl ether, 4-chloro-3,5-dimethylphenol, 1,1′-methylene-bis(3-(1-hydroxymethyl-2,4-dioximidazolidin-5-yl)urea), poly-(hexamethylenediguanide)hydrochloride, 2-phenoxyethanol, hexamethylene-tetramine, 1-(3-chloroallyl)-3,5,7-triaza-1-azonia-adamantane chloride, 1-(4-chlorophenoxy)1(1H-imidazol-1-yl)-3,3-dimethyl-2-butanone, 1,3-bis-(hydroxy-methyl)-5,5-dimethyl-2,4-imidazolidinedione, benzyl alcohol, Octopirox, 1,2-dibromo-2,4-dicyanobutane, 2,2′-methylene-bis(6-bromo-4-chlorophenol), bromochlorophene, mixture of 5-chloro-2-methyl-3(2H)-isothiazolinone and 2-methyl-3(2H)-isothiazolinone with magnesium chloride and magnesium nitrate, 2-benzyl-4-chlorophenol, 2-chloroacetamide, chlorhexidine, chlorhexidine acetate, chlorhexidine gluconate, chlorhexidine hydrochloride, 1-phenoxy-propan-2-ol, N-alkyl(C₁₂-C₂₂)trimethyl-ammonium bromide and chloride, 4,4-dimethyl-1,3-oxazolidine, N-hydroxymethyl-N-(1,3-di(hydroxymethyl)-2,5-dioxoimidazolidin-4-yl)-N′-hydroxy-methylurea, 1,6-bis(4-amidino-phenoxy)-n-hexane and its salts, glutaraldehyde, 5-ethyl-1-aza-3,7-dioxabicyclo(3.3.0)octane, 3-(4-chlorophenoxy)-1,2-propanediol, hyamines, alkyl-(C₈-C₁₈)-dimethyl-benzyl-ammonium chloride, alkyl-(C₈-C₁₈)-dimethyl-benzylammonium bromide, alkyl-(C₈-C₁₈)-dimethyl-benzyl-ammonium saccharinate, benzyl hemiformal, 3-iodo-2-propynyl butylcarbamate or sodium hydroxymethyl-aminoacetate.

(Metal) chelators which are preferably to be employed here are, inter alia, α-hydroxy fatty acids, phytic acid, lactoferrin, α-hydroxy acids and their salts, such as, inter alia, citric acid or malic acid, as well as galactaric acid, galacturonic acid, gluconic acid, glucuronic acid, ribonic acid and salts thereof, humic acids, bile acids, bile extracts, bilirubin, biliverdin or EDTA, EGTA and derivatives thereof.

Combination with Animal and/or Plant Protein Hydrolysates:

1,2-Decanediol can advantageously also be added to animal and/or plant protein hydrolysates. Substances which are advantageous in this respect are, in particular, fractions of elastin, collagen, keratin, milk protein, soya protein, oat protein, pea protein, almond protein and wheat protein, or corresponding protein hydrolysates, and also condensation products thereof with fatty acids and quaternized protein hydrolysates, the use of plant protein hydrolysates being preferred.

Combination with Solvents:

If a cosmetic and/or dermatological formulation comprising 1,2-decanediol is a solution or lotion, solvents can be chosen from the group comprising or consisting of:
water or aqueous solutions;
fatty oils, fats, waxes and other natural and synthetic fat substances, preferably esters of fatty acids with alcohols of low C number, e.g. with isopropanol, propylene glycol or glycerin, or esters of fatty alcohols with alkanoic acids of low C number or with fatty acids;
alcohols, diols or polyols of low C number, and ethers thereof, preferably isopropanol, propylene glycol, glycerin, ethylene glycol, ethylene glycol monoethyl or monobutyl ether, propylene glycol monomethyl, monoethyl or monobutyl ether, diethylene glycol monomethyl or monoethyl ether and analogous products.

Mixtures of the abovementioned solvents can preferably be used. Water can be a further constituent here.

Combination with Antioxidants:

Cosmetic formulations which comprise 1,2-decanediol can also comprise antioxidants, it being possible for all the antioxidants which are suitable or usual for cosmetic and/or dermatological uses to be used.

Combination with Vitamins:

Cosmetic formulations which comprise 1,2-decanediol can also comprise vitamins and vitamin precursors, it being possible for all the vitamins or vitamin precursors which are suitable or usual for cosmetic and/or dermatological uses to be used.

Combination with Skin Lighteners:

In numerous cases 1,2-decanediol can advantageously be employed in combination with skin lightening active compounds. According to the invention, all the skin lightening active compounds which are suitable or usual for cosmetic and/or dermatological uses can be used here. Advantageous skin lightening active compounds in this respect are kojic acid (5-hydroxy-2-hydroxymethyl-4-pyranone), kojic acid derivatives, such as e.g. kojic acid dipalmitate, arbutin, ascorbic acid, ascorbic acid derivatives, hydroquinone, hydroquinone derivatives, resorcinol, sulfur-containing molecules, such as e.g. glutathione or cysteine, alpha-hydroxy acids (e.g. citric acid, lactic acid, malic acid) and derivatives thereof, N-acetyl-tyrosine and derivatives, undecenoylphenylalanine, gluconic acid, 4-alkylresorcinols, diphenylmethane derivatives, such as e.g. 4-(1-phenylethyl)-1,3-benzenediol, chromone derivatives, such as aloesin, flavonoids, thymol derivatives, 1-aminoethylphosphinic acid, thiourea derivatives, ellagic acid, nicotinamide, zinc salts, such as e.g. zinc chloride or gluconate, thujaplicin and derivatives, triterpenes, such as maslic acid, sterols, such as ergosterol, benzofuranones, such as senkyunolide, vinyl- and ethylguiacol, inhibitors of nitrogen oxide synthesis, such as e.g. L-nitroarginine and derivatives thereof, 2,7-dinitroindazole or thiocitrullin, metal chelators (e.g. a-hydroxy-fatty acids, palmitic acid, phytic acid, lactoferrin, humic acid, bile acid, bile extracts, bilirubin, biliverdin, EDTA, EGTA and derivatives thereof), retinoids, soya milk, serine protease inhibitors or liponic acid or other synthetic or natural active compounds for lightening of the skin and hair, the latter also being used in the form of an extract from plants, such as e.g. bearberry extract, rice extract, liquorice root extract or constituents concentrated therefrom, such as glabridin or licochalcone A, Artocarpus extract, extract from Rumex and Ramulus species, extracts from pine species (Pinus) and extracts from Vitis species or stilbene derivatives concentrated therefrom, and extract from Saxifraga, mulberry, Scutelleria or/and grape.

Combination with Skin Tanning Agents:

Cosmetic formulations which comprise 1,2-decanediol can also comprise active compounds having a skin tanning action. All the skin tanning active compounds which are suitable or usual for cosmetic and/or dermatological uses can be used in this respect. Dihydroxyacetone (DHA; 1,3-dihydroxy-2-propanone) may be mentioned here by way of example. DHA can be both in the monomeric and in the dimeric form, the content of dimers predominating in the crystalline form.

Combination with Saccharides:

Cosmetic formulations which comprise 1,2-decanediol can also comprise mono-, di- and oligosaccharides, such as, for example, glucose, galactose, fructose, mannose, laevulose and lactose.

Combination with Plant Extracts:

Cosmetic formulations which comprise 1,2-decanediol can also comprise plant extracts, which are conventionally prepared by extraction of the whole plant, but also in individual cases exclusively from blossom and/or leaves, wood, bark or roots of the plant.

Combination with Surfactants:

Cosmetic formulations which comprise 1,2-decanediol can also comprise anionic, cationic, nonionic and/or amphoteric surfactants, especially if crystalline or microcrystalline solids, for example inorganic micropigments, are to be incorporated into the formulations. Surfactants are amphiphilic substances which can dissolve organic, non-polar substances in water. In this context, the hydrophilic contents of a surfactant molecule are usually polar functional groups, for example —COO⁻, —OSO₃²⁻ or —SO₃⁻, while the hydrophobic parts as a rule are non-polar hydrocarbon radicals. Surfactants are in general classified according to the nature and charge of the hydrophilic molecular moiety. A distinction can be made between four groups here:
anionic surfactants,
cationic surfactants,
amphoteric surfactants and
nonionic surfactants.

Anionic surfactants as a rule contain carboxylate, sulfate or sulfonate groups as functional groups. In aqueous solution, they form negatively charged organic ions in an acid or neutral medium. Cationic surfactants are almost exclusively characterized by the presence of a quaternary ammonium group. In aqueous solution, they form positively charged organic ions in an acid or neutral medium. Amphoteric surfactants contain both anionic and cationic groups and accordingly behave like anionic or cationic surfactants in aqueous solution, depending on the pH. In a strongly acid medium they have a positive charge, and in an alkaline medium a negative charge. On the other hand, they are zwitter-ionic in the neutral pH range. Polyether chains are typical of nonionic surfactants. Nonionic surfactants do not form ions in an aqueous medium.

A. Anionic Surfactants

Anionic surfactants which are advantageously to be used are acylamino acids (and salts thereof), such as
acyl glutamates, for example sodium acyl glutamate, di-TEA-palmitoyl aspartate and sodium capryl/capric glutamate,
acyl peptides, for example palmitoyl hydrolysed milk protein, sodium cocoyl hydrolysed soya protein and sodium/potassium cocoyl hydrolysed collagen,
sarcosinates, for example myristoyl sarcosine, TEA-lauroyl sarcosinate, sodium lauroyl sarcosinate and sodium cocoyl sarcosinate,
taurates, for example sodium lauroyl taurate and sodium methylcocoyl taurate,
acyl lactylates, for example lauroyl lactylate, caproyl lactylated and stearoyl lactylate
alaninates

carboxylic acids and derivatives, such as

for example, lauric acid, aluminium stearate, magnesium alkanolate and zinc undecylenate,
ester-carboxylic acids, for example calcium stearoyl lactylate, laureth-6 citrate and sodium PEG-4 lauramide carboxylate,
ether-carboxylic acids, for example sodium laureth-13 carboxylate and sodium PEG-6 cocamide carboxylate,

phosphoric acid esters and salts, such as, for example, DEA-oleth-10 phosphate and dilaureth-4 phosphate,

sulfonic acids and salts, such as
acyl isothionates, e.g. sodium/ammonium cocoyl isethionate,
alkylarylsulfonates,
alkylsulfonates, for example sodium coco-monoglyceride sulfate, sodium C₁₂₋₁₄olefin-sulfonate, sodium lauryl sulfoacetate and magnesium PEG-3 cocamide sulfate,
sulfosuccinates, for example dioctyl sodium sulfosuccinate, disodium laureth-sulfosuccinate, disodium laurylsulfosuccinate and disodium undecylenamido-MEA-sulfosuccinate

and

sulfuric acid esters, such as
alkyl ether-sulfate, for example sodium, ammonium, magnesium, MIPA and TIPA laureth sulfate, sodium myreth sulfate and sodium C12-13 pareth sulfate,
alkyl sulfates, for example sodium, ammonium and TEA lauryl sulfate.

B. Cationic Surfactants

Cationic surfactants which are advantageously to be used are
alkylamines,
alkylimidazoles,
ethoxylated amines and
quaternary surfactants,

RNH₂CH₂CH₂COO⁻ (at pH=7)

RNHCH₂CH₂COO—B⁺ (at pH=12) B⁺=any desired cation, e.g. Na⁺
ester quats

Quaternary surfactants contain at least one N atom which is covalently bonded to 4 alkyl or aryl groups. This leads to a positive charge, independently of the pH. Alkylbetaine, alkylamidopropylbetaine and alkylamidopropylhydroxysulfaine are advantageous. The cationic surfactants used can furthermore preferably be chosen from the group consisting of quaternary ammonium compounds, in particular benzyltrialkyl-ammonium chlorides or bromides, such as, for example, benzyldimethylstearyl-ammonium chloride, furthermore alkyltrialkylammonium salts, for example cetyltrimethylammonium chloride or bromide, alkyldimethylhydroxy-ethylammonium chlorides or bromides, dialkyldimethylammonium chlorides or bromides, alkylamide-ethyltrimethyl-ammonium ether-sulfates, alkylpyridinium salts, for example lauryl- or cetylpyrimidinium chloride, imidazoline derivatives and compounds having a cationic character, such as amine oxides, for example alkyldimethylamine oxides or alkylaminoethyldimethylamine oxides. Cetyltrimethyl-ammonium salts in particular are advantageously to be used.

C. Amphoteric Surfactants

Amphoteric surfactants which are advantageously to be used are
acyl-/dialkylethylenediamine, for example sodium acylamphoacetate, disodium acylamphodipropionate, disodium alkylamphodiacetate, sodium acyl-amphohydroxy-propylsulfonate, disodium acylamphodiacetate and sodium acylamphopropionate,
N-alkylamino acids, for example aminopropyl alkylglutamide, alkylaminopropionic acid, sodium alkylimidodipropionate and lauroamphocarboxyglycinate.

D. Nonionic Surfactants

Nonionic surfactants which are advantageously to be used are
alcohols,
alkanolamides, such as cocamides MEA/DEA/MIPA,
amine oxides, such as cocoamidopropylamine oxide,
esters which are formed by esterification of carboxylic acids with ethylene oxide, glycerin, sorbitan or other alcohols,
ethers, for example ethoxylated/propoxylated alcohols, ethoxylated/propoxylated esters, ethoxylated/propoxylated glycerin esters, ethoxylated/propoxylated cholesterols, ethoxylated/propoxylated triglyceride esters, ethoxylated/propoxylated lanolin, ethoxylated/propoxylated polysiloxanes, propoxylated POE ethers and alkyl polyglycosides, such as lauryl glucoside, decyl glycoside and coconut glycoside.
sucrose esters and ethers
polyglycerin esters, diglycerin esters and monoglycerin esters
methylglucose esters and esters of hydroxy acids

The use of a combination of anionic and/or amphoteric surfactants with one or more nonionic surfactants is furthermore advantageous.

The surface-active substance can be present in a concentration of between 1 and 98 wt. % in the formulations comprising 1,2-decanediol, based on the total weight of the formulations.

Emulsions comprising a mixture according to the invention:

Cosmetic or dermatological formulations which comprise 1,2-decanediol can also be in the form of emulsions.

The oily phase can advantageously be chosen from the following substance group:
mineral oils and mineral waxes
fatty oils, fats, waxes and other natural and synthetic fat substances, preferably esters of fatty acids with alcohols of low C number, e.g. with isopropanol, propylene glycol or glycerin, or esters of fatty alcohols with alkanoic acids of low C number or with fatty acids;
alkyl benzoates;
silicone oils, such as dimethylpolysiloxanes, diethylpolysiloxanes, diphenylpolysiloxanes and mixed forms thereof.

Compounds which can advantageously be employed are non-comedogenically active esters of saturated and/or unsaturated branched and/or unbranched alkanecarboxylic acids having a chain length of from 3 to 30 C atoms and saturated and/or unsaturated, branched and/or unbranched alcohols having a chain length of from 3 to 30 C atoms. Preferred ester oils are 3,5,5-trimethylhexyl 3,5,5-trimethylhexanoate, 2-ethylhexyl isononanoate, 2-ethylhexyl 3,5,5-trimethylhexanoate, 2-ethylhexyl 2-ethylhexanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of such esters, e.g. jojoba oil.

In addition, however, esters of aromatic carboxylic acids and saturated and/or unsaturated, branched and/or unbranched alcohols having a chain length of from 3 to 30 C atoms can also be advantageously combined with 1 ,2-decanediol.

The oily phase can furthermore advantageously be chosen from the group consisting of branched and unbranched hydrocarbons and hydrocarbon waxes, silicone oils and dialkyl ethers. the group consisting of saturated or unsaturated, branched or unbranched alcohols, and the fatty acid triglycerides, namely the triglycerin esters of saturated and/or unsaturated, branched and/or unbranched alkanecarboxylic acids having a chain length of from 8 to 24, in particular 12 to 18 C atoms. The fatty acid triglycerides can advantageously be chosen from the group consisting of synthetic, semi-synthetic and natural oils, e.g. olive oil, sunflower oil, soya oil, groundnut oil, rapeseed oil, almond oil, palm oil, coconut oil, palm kernel oil and more of the like. Any desired blends of such oil and wax components can also advantageously be employed. In some cases it is also advantageous to employ waxes, for example cetyl palmitate, as the sole lipid component of the oily phase, and the oily phase is advantageously chosen from the group which consists of 2-ethylhexyl isostearate, octyldodecanol, isotridecyl isononanoate, isoeicosane, 2-ethylhexyl cocoate, C₁₂₋₁₅-alkyl benzoate, caprylic/capric acid triglyceride and dicaprylyl ether. Mixtures of C₁₂₋₁₅-alkyl benzoate and 2-ethylhexyl isostearate, mixtures of C₁₂₋₁₅-alkyl benzoate and isotridecyl isononanoate and mixtures of C₁₂₋₁₅-alkyl benzoate, 2-ethylhexyl isostearate and isotridecyl isononanoate are particularly advantageous. The hydrocarbons paraffin oil, squalane and squalene can also advantageously be used. The oily phase can furthermore have a content of cyclic or linear silicone oils or consist entirely of such oils, it nevertheless being preferable to use an additional content of other oily phase components in addition to the silicone oil or silicone oils. Cyclomethicone (e.g. decamethylcyclopentasiloxane) can advantageously be employed as a silicone oil. However, other silicone oils, for example undecamethylcyclotrisiloxane, polydimethylsiloxane and poly(methyl-phenylsiloxane), can also advantageously be used. Mixtures of cyclomethicone and isotridecyl isononanoate and of cyclomethicone and 2-ethylhexyl isostearate are furthermore particularly advantageous.

The aqueous phase of formulations which comprise 1,2-decanediol and are in the form of an emulsion can advantageously comprise: alcohols, diols or polyols of low C number and ethers thereof, preferably ethanol, isopropanol, propylene glycol, glycerin, ethylene glycol, ethylene glycol monoethyl or monobutyl ether, propylene glycol monomethyl, monoethyl or monobutyl ether, diethylene glycol monomethyl or monoethyl ether and analogous products, and furthermore alcohols of low C number, e.g. ethanol, isopropanol, 1,2-propanediol and glycerin, and, in particular, one or more thickening agents, which can advantageously be chosen from the group consisting of silicon dioxide, aluminium silicates, polysaccharides and derivatives thereof, e.g. hyaluronic acid, xanthan gum and hydroxypropyl-methylcellulose, particularly advantageously from the group consisting of polyacrylates, preferably a polyacrylate from the group consisting of the so-called Carbopols, for example Carbopols of the types 980, 981, 1382, 2984 and 5984, in each case individually or in combination.

Formulations which comprise 1,2-decanediol and are in the form of an emulsion advantageously comprise one or more emulsifiers. O/W emulsifiers can advantageously be chosen, for example, from the group consisting of polyethoxylated or polypropoxylated or polyethoxylated and polypropoxylated products, e.g.:
the fatty alcohol ethoxylates
the ethoxylated wool wax alcohols
the polyethylene glycol ethers of the general formula R—O—(—CH₂—CH₂—O—)ₙ—R′,
the fatty acid ethoxylates of the general formula R—COO—(—CH₂—CH₂—O—)ₙ—H,
the etherified fatty acid ethoxylates of the general formula

R—COO—(—CH₂—CH₂—O—)ₙ—R′,
the esterified fatty acid ethoxylates of the general formula

R—COO—(—CH₂—CH₂—O—)ₙ—C(O)—R′,
the polyethylene glycol glycerin fatty acid esters
the ethoxylated sorbitan esters
the cholesterol ethoxylates
the ethoxylated triglycerides
the alkyl ether-carboxylic acids of the general formula

R—COO—(—CH₂—CH₂—O—)ₙ—OOH, wherein n represents a number from 5 to 30,
the polyoxyethylene sorbitol fatty acid esters
the alkyl ether-sulfates of the general formula R—O—(—CH₂—CH₂—O—)ₙ—SO₃—H
the fatty alcohol propoxylates of the general formula R—O—(—CH₂—CH(CH₃)—O—)ₙ—H
the polypropylene glycol ethers of the general formula

R—O—(—CH₂—CH(CH₃)—O—)ₙ—R′
the propoxylated wool wax alcohols,
the etherified fatty acid propoxylates R—COO—(—CH₂—CH(CH₃)—O—)ₙ—R′
the esterified fatty acid propoxylates of the general formula

R—COO—(—CH₂—CH(CH₃)—O—)ₙ—C(O)—R′
the fatty acid propoxylates of the general formula

R—COO—(—CH₂—CH(CH₃)—O—)ₙ—H,
the polypropylene glycol glycerin fatty acid esters
the propoxylated sorbitan esters
the cholesterol propoxylates
the propoxylated triglycerides
the alkyl ether-carboxylic acids of the general formula

R—O—(—CH₂—CH(CH₃)—O—)ₙ—CH₂—COOH,
the alkyl ether-sulfates and the acids on which these sulfates are based of the general formula R—O—(—CH₂—CH(CH₃)—O—)ₙ—SO₃—H,
the fatty alcohol ethoxylates/propoxylates of the general formula R—O—Xₙ—Yₘ—H
the polypropylene glycol ethers of the general formula R—O—Xₙ—Yₘ—R′
the etherified fatty acid propoxylates of the general formula R—COO—Xₙ—Yₘ—R′
the fatty acid ethoxylates/propoxylates of the general formula R—COO—Xₙ—Yₘ—H.

According to the invention, the polyethoxylated or polypropoxylated or polyethoxylated and polypropoxylated O/W emulsifiers employed are particularly advantageously chosen from the group consisting of substances having HLB values of 11-18, very particularly advantageously having HLB values of 14.5-15.5, if the O/W emulsifiers contain saturated radicals R and R′. If the O/W emulsifiers contain unsaturated radicals R and/or R′, or isoalkyl derivatives are present, the preferred HLB value of such emulsifiers can also be lower or higher.

It is of advantage to choose the fatty alcohol ethoxylates from the group consisting of ethoxylated stearyl alcohols, cetyl alcohols and cetylstearyl alcohols (cetearyl alcohols). The following are particularly preferred:

polyethylene glycol(13) stearyl ether (steareth-13), polyethylene glycol(14) stearyl ether (steareth-14), polyethylene glycol(15) stearyl ether (steareth-15), polyethylene glycol(16) stearyl ether (steareth-16), polyethylene glycol(17) stearyl ether (steareth-17), polyethylene glycol(18) stearyl ether (steareth-18), polyethylene glycol(19) stearyl ether (steareth-19), polyethylene glycol(20) stearyl ether (steareth-20), polyethylene glycol(12) isostearyl ether (isosteareth-12), polyethylene glycol(13) isostearyl ether (isosteareth-13), polyethylene glycol(14) isostearyl ether (isosteareth-14), polyethylene glycol(15) isostearyl ether (isosteareth-15), polyethylene glycol(16) isostearyl ether (isosteareth-16), polyethylene glycol(17) isostearyl ether (isosteareth-17), polyethylene glycol(18) isostearyl ether (isosteareth-18), polyethylene glycol(19) isostearyl ether (isosteareth-19), polyethylene glycol(20) isostearyl ether (isosteareth-20), polyethylene glycol(13) cetyl ether (ceteth-13), polyethylene glycol(14) cetyl ether (ceteth-14), polyethylene glycol(15) cetyl ether (ceteth-15), polyethylene glycol(16) cetyl ether (ceteth-16), polyethylene glycol(17) cetyl ether (ceteth-17), polyethylene glycol(18) cetyl ether (ceteth-18), polyethylene glycol(19) cetyl ether (ceteth-19), polyethylene glycol(20) cetyl ether (ceteth-20), polyethylene glycol(13) isocetyl ether (isoceteth-13), polyethylene glycol(14) isocetyl ether (isoceteth-14), polyethylene glycol(15) isocetyl ether (isoceteth-15), polyethylene glycol(16) isocetyl ether (isoceteth-16), polyethylene glycol(17) iso-cetyl ether (isoceteth-17), polyethylene glycol(18) isocetyl ether (isoceteth-18), polyethylene glycol(19) isocetyl ether (isoceteth-19), polyethylene glycol(20) isocetyl ether (isoceteth-20), polyethylene glycol(12) oleyl ether (oleth-12), polyethylene glycol(13) oleyl ether (oleth-13), polyethylene glycol-(14) oleyl ether (oleth-14), polyethylene glycol(15) oleyl ether (oleth-15), polyethylene glycol(12) lauryl ether (laureth-12), polyethylene glycol(12) isolauryl ether (isolaureth-12), polyethylene glycol(13) cetylstearyl ether (ceteareth-13), poly-ethylene glycol(14) cetylstearyl ether (ceteareth-14), polyethylene glycol(15) cetyl-stearyl ether (ceteareth-15), polyethylene glycol(16) cetylstearyl ether (ceteareth-16), polyethylene glycol(17) cetylstearyl ether (ceteareth-17), polyethylene glycol-(18) cetylstearyl ether (ceteareth-18), polyethylene glycol(19) cetylstearyl ether (ceteareth-19) and polyethylene glycol(20) cetylstearyl ether (ceteareth-20).

It is furthermore advantageous to choose the fatty acid ethoxylates from the following group:

polyethylene glycol(20) stearate, polyethylene glycol(21) stearate, polyethylene glycol(22) stearate, polyethylene glycol(23) stearate, polyethylene glycol(24) stearate, polyethylene glycol(25) stearate, polyethylene glycol(12) isostearate, polyethylene glycol(13) isostearate, polyethylene glycol-(14) isostearate, polyethylene glycol(15) isostearate, polyethylene glycol(16) isostearate, polyethylene glycol(17) isostearate, polyethylene glycol(18) isostearate, polyethylene glycol(19) isostearate, polyethylene glycol(20) isostearate, polyethylene glycol(21) isostearate, polyethylene glycol-(22) isostearate, polyethylene glycol(23) isostearate, polyethylene glycol(24) isostearate, polyethylene glycol(25) isostearate, polyethylene glycol(12) oleate, polyethylene glycol(13) oleate, polyethylene glycol(14) oleate, polyethylene glycol(15) oleate, polyethylene glycol(16) oleate, polyethylene glycol(17) oleate, polyethylene glycol(18) oleate, polyethylene glycol(19) oleate, polyethylene glycol(20) oleate.

Sodium laureth-11 carboxylate can advantageously be used as an ethoxylated alkyl ether-carboxylic acid or salt thereof. Sodium laureth 1-4 sulfate can advantageously be used as an alkyl ether-sulfate. Polyethylene glycol(30) cholesteryl ether can advantageously be used as an ethoxylated cholesterol derivative. Polyethylene glycol(25) soyasterol has also proved suitable.

The polyethylene glycol(60) evening primrose glycerides can advantageously be used as ethoxylated triglycerides.

It is furthermore advantageous to choose the polyethylene glycol glycerin fatty acid esters from the group consisting of polyethylene glycol(20) glyceryl laurate, polyethylene glycol(21) glyceryl laurate, polyethylene glycol(22) glyceryl laurate, polyethylene glycol(23) glyceryl laurate, polyethylene glycol(6) glyceryl caprate/caproate, polyethylene glycol(20) glyceryl oleate, polyethylene glycol(20) glyceryl isostearate and polyethylene glycol(18) glyceryl oleate/cocoate.

It is likewise favourable to choose the sorbitan esters from the group consisting of polyethylene glycol(20) sorbitan monolaurate, polyethylene glycol(20) sorbitan monostearate, polyethylene glycol(20) sorbitan monoisostearate, polyethylene glycol(20) sorbitan monopalmitate and polyethylene glycol(20) sorbitan monooleate.

Advantageous W/O emulsifiers which can be employed are: fatty alcohols having 8 to 30 carbon atoms, monoglycerin esters of saturated and/or unsaturated, branched and/or unbranched alkanecarboxylic acids having a chain length of from 8 to 24, in particular 12 to 18 C atoms, diglycerin esters of saturated and/or unsaturated, branched and/or unbranched alkanecarboxylic acids having a chain length of from 8 to 24, in particular 12 to 18 C atoms, monoglycerin ethers of saturated and/or unsaturated, branched and/or unbranched alcohols having a chain length of from 8 to 24, in particular 12 to 18 C atoms, diglycerin ethers of saturated and/or unsaturated, branched and/or unbranched alcohols having a chain length of from 8 to 24, in particular 12 to 18 C atoms, propylene glycol esters of saturated and/or unsaturated, branched and/or unbranched alkanecarboxylic acids having a chain length of from 8 to 24, in particular 12 to 18 C atoms and sorbitan esters of saturated and/or unsaturated, branched and/or unbranched alkanecarboxylic acids having a chain length of from 8 to 24, in particular 12 to 18 C atoms.

W/O emulsifiers which are advantageous in particular are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol(2) stearyl ether (steareth-2), glyceryl monolaurate, glyceryl monocaprate and glyceryl monocaprylate.

Combination with Sunscreen Compositions:

For use, the cosmetic and/or dermatological/keratological formulations comprising 1,2-decanediol are applied to the skin in a sufficient amount in the conventional manner for cosmetics and dermatics. In this context, cosmetic and dermatological formulations which comprise 1,2-decanediol and additionally act as sunscreen compositions offer particular advantages. These formulations advantageously comprise at least one UVA filter and/or at least one UVB filter and/or at least one inorganic pigment. In this context, the formulations can be in various forms such as are conventionally employed e.g. for this type of formulation. They can be e.g. a solution, an emulsion of the water-in-oil (W/O) type or of the oil-in-water (O/W) type or a multiple emulsion, for example of the water-in-oil-in-water (W/O/W) type, a gel, a hydrodispersion, a solid stick or also an aerosol.

Combination with Thickeners:

1,2-Decanediol can advantageously also be combined with thickeners. Suitable thickeners are: homopolymers of acrylic acid having a molecular weight of from 2,000,000 to 6,000,000. such as e.g. the commercial product Carbopols. Further thickeners are marketed under the names Carbopol 940, Carbopol EDTA 2001 or Modarez V 600 PX. Polymers of acrylic acid and acrylamide (sodium salt) are having a molecular weight of from 2,000,000 to 6,000,000, such as e.g. Hostacerin PN 73 or the sclerotium gum marketed under the name Amigel. Copolymers of acrylic acid or methacrylic acid, such as e.g. Carbopol 1342 or Permulen TRI, are moreover suitable. Further thickener types are polyglycols, cellulose derivatives, in particular hydroxyalkylcelluloses, and alginates, carrageenan and inorganic thickeners, such as e.g. natural or synthetic bentonites.

Combination with Fillers:

1,2-Decanediol can further advantageously, although not necessarily, be combined with fillers which e.g. further improve the sensorial and cosmetic properties of the formulations and, for example, bring about or intensify a velvety or silky sensation on the skin. Advantageous fillers in the context of the present invention are starch and starch derivatives (such as e.g. tapioca starch, distarch phosphate, aluminium- or sodium-starch octenylsuccinate and the like), pigments which have neither a chiefly UV filter nor a colouring action (such as e.g. boron nitride etc.) and/or Aerosils® (CAS no. 7631-86-9).

Combination with Cosmetic Auxiliary Substances:

In cosmetic formulations, 1,2-decanediol can advantageously also be combined with cosmetic auxiliary substances such as are conventionally used in such formulations, e.g. antioxidants, perfume oils, agents for preventing foaming, dyestuffs, pigments which have a colouring action, thickeners, surface-active substances, emulsifiers, softening substances, further moisturizing and/or moisture-retaining substances, fats, oils, waxes or other conventional constituents of a cosmetic formulation, such as alcohols, polyols, polymers, foam stabilizers, electrolytes, organic solvents or silicone derivatives. According to the invention, all conceivable antioxidants, perfume oils, agents for preventing foaming, dyestuffs, pigments which have a colouring action, thickeners, surface-active substances, emulsifiers, softening substances, moisturizing and/or moisture-retaining substances, fats, oils, waxes, alcohols, polyols, polymers, foam stabilizers, electrolytes, organic solvents or silicone derivatives which are suitable or usual for cosmetic and/or dermatological uses can be used here.

Combination with Odoriferous Substances:

1,2-Decanediol can also be employed as a constituent of fragrance compositions (odoriferous substance compositions) for hair and scalp care products, and in particular due to its specific activity, for example, can impart to a perfumed finished product an additional sebum-reducing and anti-acne action. Particularly preferred fragrance compositions comprise (a) a sensorially active amount of an odoriferous substance or of the sum of two, three or more odoriferous substances, (b) an amount of 1,2-decanediol which imparts a sebum-reducing action and (c) optionally one or more carrier substances and/or additives. Since the content of perfume in a cosmetic finished product is often in the region of approx. 1 wt. %, a perfume which contains 1,2-decanediol preferably comprises 1,2-decanediol to the extent of about 0.1-10 wt. %. It has proved to be particularly advantageous that 1,2-decanediol has only a weak intrinsic smell or is even completely odourless, since this property predestines it in particular for use in a fragrance composition.

Combination with Dyestuffs and/or Coloured Pigments:

Cosmetic and dermatological formulations which comprise 1,2-decanediol can comprise dyestuffs and/or coloured pigments, especially if they are in the form of decorative cosmetics. The dyestuffs and coloured pigments can be chosen from the corresponding positive list of cosmetics legislation or the EU list of cosmetic colouring agents. In most cases, they are identical to the dyestuffs approved for foodstuffs. Advantageous coloured pigments are, for example, titanium dioxide, mica, iron oxides (e.g. Fe2O3. Fe3O4, FeO(OH)) and/or tin oxide. Advantageous dyestuffs are, for example, carmine, Berlin blue, chromium oxide green, ultramarine blue and/or manganese violet. It is particularly advantageous to choose the dyestuffs and/or coloured pigments from the Rowe Colour Index, 3rd edition, Society of Dyers and Colourists, Bradford, England, 1971.

If the formulations comprising 1,2-decanediol are in the form of products which are used on the face, it is favourable to choose as the dyestuff one or more substances from the following group: 2,4-dihydroxyazobenzene, 1-(2′-chloro-4′-nitro-1′-phenylazo)-2-hydroxynaphthalene, Ceres Red, 2-(sulfo-1-naphthylazo)-1-naphthol-4-sulfonic acid, calcium salt of 2-hydroxy-1,2′-azonaphthalene-1′-sulfonic acid, calcium and barium salts of 1-(2-sulfo-4-methyl-1-phenylazo)-2-naphthylcarboxylic acid, calcium salt of 1-(2-sulfo-1-naphthylazo)-2-hydroxynaphthalene-3-carboxylic acid, aluminium salt of 1-(4-sulfo-1-phenylazo)-2-naphthol-6-sulfonic acid, aluminium salt of 1-(4-sulfo-1-naphthylazo)-2-naphthol-3,6-disulfonic acid, 1-(4-sulfo-1-naphthylazo)-2-naphthol-6,8-disulfonic acid, aluminium salt of 4-(4-sulfo-1-phenylazo)-1-(4-sulfophenyl)-5-hydroxy-pyrazolone-3-carboxylic acid, aluminium and zirconium salts of 4,5-dibromofluorescein, aluminium and zirconium salts of 2,4,5,7-tetrabromofluorescein, 3′,4′,5′,6′-tetrachloro-2,4,5,7-tetrabromofluorescein and its aluminium salt, aluminium salt of 2,4,5,7-tetraiodofluorescein, aluminium salt of quinophthalone-disulfonic acid, aluminium salt of indigo-disulfonic acid, red and black iron oxide (CIN: 77 491 (red) and 77 499 (black)), iron oxide hydrate (CIN: 77 492), manganese ammonium diphosphate and titanium dioxide.

Oil-soluble natural dyestuffs, such as e.g. paprika extracts, β-carotene or cochineal, are furthermore advantageous. Formulations having a content of pearlescent pigments are furthermore advantageous.

In this context, 1,2-decanediol can be incorporated without difficulties into the usual cosmetic or dermatological/keratological formulations according to the invention, such as, inter alia, pump sprays, aerosol sprays, creams, ointments, tinctures, lotions, cleansing emulsions, cleansing milk, cream-like washing emulsions, cleansing washing gels, cleansing and/or care facial waters, cleansing cloths and, in the scalp area, also into shampoos, hair treatment bases and the like. It is also possible here, and in some cases advantageous, to combine 1,2-decanediol with further active compounds. The cosmetic and/or dermatological/keratological formulations according to the invention comprising 1,2-decanediol can otherwise have the conventional composition here and serve for treatment of the skin in the sense of a dermatological/keratological treatment or a treatment in the sense of care cosmetics. However, 1,2-decanediol can moreover also be employed in make-up products in decorative cosmetics.

Further preferred embodiments of the mixtures according to the invention and of the methods and uses according to the invention can be seen from the following examples and the associated tables.

### EMBODIMENT EXAMPLES

The following examples are intended to illustrate the present invention without limiting it. All the amounts data, contents and percentage contents are, unless otherwise stated, based on the weight and the total amount or on the total weight of the formulations.

### Example 1

Lowering of the Dropping Point of a Model Sebum Containing 1,2-Decanediol

To check the extent to which 1,2-decanediol has a sebum-fluidizing property and is therefore capable of reducing the sebum concentration on the skin surface, and in particular in the sebaceous gland follicles, by a physical route, the influence of 1,2-decanediol on the dropping point of a model sebum was first investigated.

The following analyses of the composition of human sebum described in the literature were used for preparation of the model sebum:

(A) “Differential scanning calorimetry studies of sebum models”, J. Cosmet. Sci., 52, 211-224 Average composition of human skin surface lipids for 17 subjects);

(B) “Das seborrhoische Ekzem-Ein Spannungsfeld in der Dermatologie [Seborrhoeic eczema—A stress field in dermatology]” Uniklinik Münster Prof. Dr. med T. A. Luger, Dissertation Frau Kathrin Decker 2004;

(C) “The role of sebaceous gland activity and scalp microfloral metabolism in the etiology of seborrhoic dermatitis and dandruff”, P&G, Beauty Care Technology Division+Department of Dermatology Chung Ang University, Seoul Korea

On the basis of these publications, a model serum was developed which substantially comprises saturated and unsaturated free fatty acids, wax-like substances, triglycerides and cholesterol and, in detail, is composed of the following substances: palmitic acid, oleic acid, stearic acid, squalene, myristyl myristate, cetyl palmitate, tripalmitin, triolein and cholesterol. The exact quantitative composition of the model sebum (contents of the individual substances in per cent by weight; wt. %) can be seen from the following Table 1.

TABLE 1
Qualitative and quantitative composition of model sebum

| | |
|---|---|
| Model sebum | Wt. % |

Free fatty acids, 22%

| | |
|---|---|
| Palmitic acid | 8.0 |
| Oleic acid | 10.0 |
| Stearic acid | 4.0 |

Squalenes 12%

| | |
|---|---|
| Squalene | 12.0 |

Wax esters 24%

| | |
|---|---|
| Myristyl myristate | 12.0 |
| Cetyl palmitate | 12.0 |

Triglycerides, 41%

| | |
|---|---|
| Tripalmitin | 15.0 |
| Triolein | 26.0 |

Cholesterol

| | |
|---|---|
| Cholesterol | 1.0 |
| Total | 100.0 |

To investigate the influence of 1,2-decanediol on the physical properties of the model sebum, 20 wt. % 1,2-decanediol was added to the model sebum. The dropping points of the 1,2-decanediol-free model sebum and the model sebum sample containing 20 wt. % 1,2-decanediol were then determined using the method for dropping point determination according to AOCS Cc 18-80/ASTM D 3954.

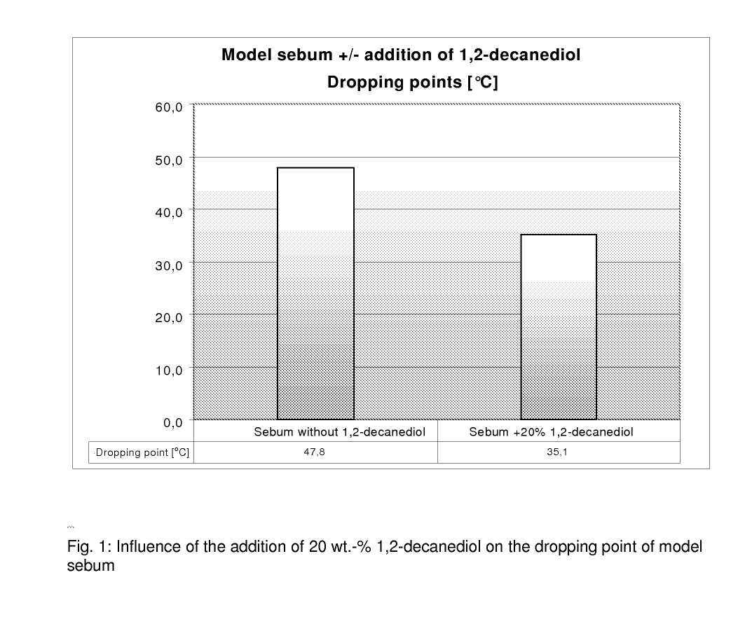

The results of the investigations are shown in a graph in FIG. 1.

The investigations clearly show that the dropping point of model sebum is reduced significantly by 12.7° C. by the addition of 20 wt. % 1,2-decanediol. It is thus demonstrated that 1,2-decanediol contributes towards the liquefaction of sebum.

### Example 2

Human in vivo Study to Demonstrate the Reduction of the Sebum Concentration of the Skin by a Cosmetic Formulation Comprising 2% 1,2-Decanediol

To test whether the reduction in the dropping point of a model sebum and the associated increased sebum fluidization determined in the in vitro experiment has a clinical relevance and the sebum concentration on the skin surface can also be reduced by this specific physical effect in a human in vivo situation, a clinical study was conducted on male test subjects with greasy-oily and/or impure skin.

The parameters of the experiment are described in detail below:

Number of test subjects: 20 male persons aged 17-19 years

Test area: forehead (half-side test—placebo versus active compound formulation)

Test duration: 28 days

Test products:

GS06016-1 (placebo formulation without 1,2-decanediol)

GS06016-2 (formulation comprising 2% 1,2-decanediol)

Gel Formulations:

| | INCI | GS06016-1 | GS06016-2 |
|---|---|---|---|
| ETD 2050 0.5% | Carbomer | 98.2 | 96.2 |
| dispersion | | | |
| 1,2-Decanediol | 1,2 Decane diol | — | 2.0 |
| Sodium hydroxide | Sodium | 1.0 | 1.0 |
| solution, 10% strength | hydroxide | | |
| Dragocid Liquid | Phenoxyethanol, | 0.8 | 0.8 |
| | methyl- | | |
| | paraben, | | |
| | ethylparaben, | | |
| | butylparaben, | | |
| | propylparaben, | | |
| | isobutylparaben | | |

Frequency of use: 2 times daily (morning and evening) according to conventional daily care practice

Evaluation criteria:

a) Sebum content (Sebumetry; Sebumeter ®SM 810;Courage & Khazaka, Cologne, Germany).

b) Skin purity/skin condition (number/intensity of pimples and pustules—visual evaluation)

Measurement points: 0, 14, 21 and 28 days

Result

a) Sebum Content (Sebumetry; Sebumeter®SM 810;Courage & Khazaka, Köln, Germany).

As the human in vivo study showed, when the product GS06016-2 (formulation comprising 2% 1,2-decanediol) was used in a direct comparison (half-side test/left versus right half of forehead) it was possible to measure a statistically significant (p<0.05) reduction in the sebum content compared with GS06016-1 (placebo formulation without 1,2-decanediol) both after the period t=21 days (reduction in the sebum content by 19.8 μg/cm², based on the initial sebum content at time=0) and after the period t=28 days (reduction in the sebum content by 29.4 μg/cm², based on the initial sebum content at time t=0).

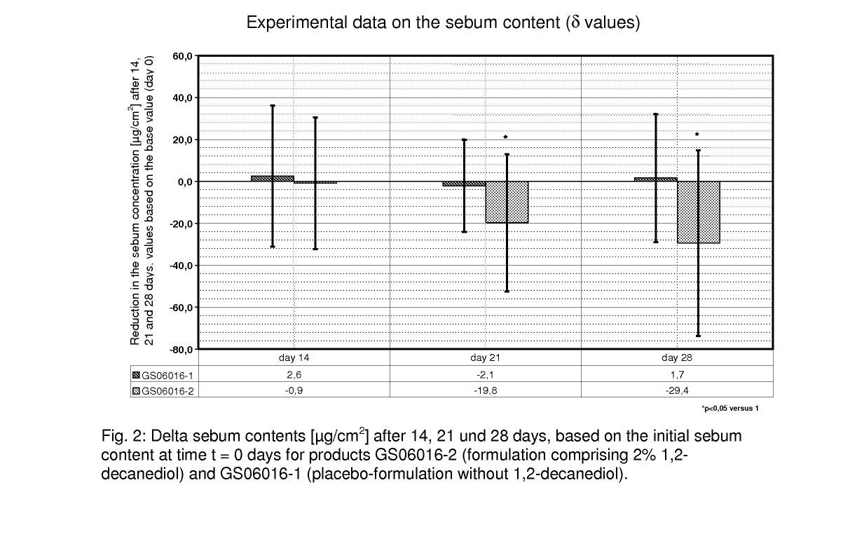

In contrast, no reduction in the sebum content was to be detected for product GS06016-1 (placebo formulation without 1,2-decanediol). The results of the sebum reduction (delta values in each case based on the sebum content at time t=0 days) are shown on a graph in FIG. 2 for products GS06016-1 and GS06016-2.

b) Skin Purity/Skin Condition (Number of Pimples and Pustules—Visual Evaluation)

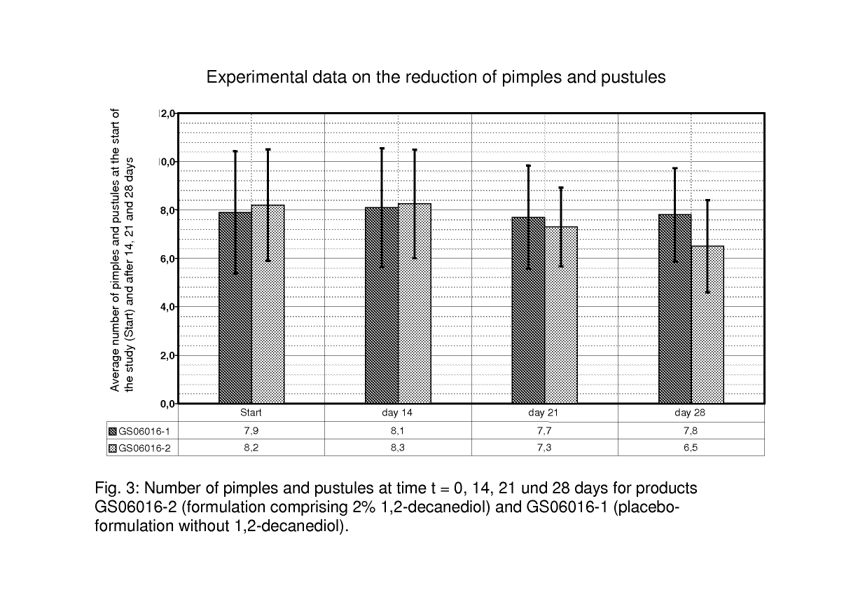

As the human in vivo study likewise showed, when product GS06016-1 (formulation comprising 2% 1,2-decanediol) was used in a direct comparison (half-side test/left versus right half of forehead), it was possible to achieve a clear reduction in the number of pimples and pustules compared with the starting value (average number of pimples and pustules: 8.2) after the period t=28 days (average number of pimples and pustules: 6.5). In contrast, no reduction was detectable for the formulation GS06016-1 (placebo formulation without 1,2-decanediol). The results are shown on a graph in FIG. 3.

These results show that a significant reduction of the sebum concentration of the skin is achieved due to the use of 1,2-decanediol.

### Example 3

### Formulation Examples

| Raw material | INCI | 1 | 2 | 3 | 4 | 5 | 6 | 7 | 8 |
|---|---|---|---|---|---|---|---|---|---|
| (−)-alpha-Bisabolol, natural | Bisabolol | | | 0.10 | | | | | |
| (Symrise) | | | | | | | | | |
| Abil 350 | Dimethicone | | | | 1.00 | | | | 0.50 |
| Allantoin | Allantoin | 0.10 | 0.20 | | | 0.10 | | | |
| Azelaic acid | Azelaic acid | | | | | | 20.00 | | |
| Benzoyl peroxide | Benzoyl peroxide | | | | 10.00 | | | | |
| Carbopol EDT 2050 | Carbomer | | | 0.10 | | 1.00 | | | |
| Carbopol Ultrez 21 | Acrylates/C10-30 Alkyl | | | | | | 0.25 | | |
| | Acrylate Crosspolymer | | | | | | | | |
| Citric acid, 10% strength | Citric Acid | | 0.30 | | 0.35 | | | | |
| Clindamycin | | | | | 3.0 | | | | |
| Cremogen M-82 | Water(Aqua), Propylene | | | | 1.50 | | | | |
| | Glycol, Ethoxydiglycol, | | | | | | | | |
| | Glucose, Aesculus | | | | | | | | |
| | Hippocastanum (Horse | | | | | | | | |
| | Chestnut) Seed Extract, | | | | | | | | |
| | Salvia Officinalis (Sage) | | | | | | | | |
| | Leaf Extract, Equisetum | | | | | | | | |
| | Arvense Extract, Tussilago | | | | | | | | |
| | Farfara (Coltsfoot) Leaf | | | | | | | | |
| | Extract, Rosmarinus | | | | | | | | |
| | Officinalis (Rosemary) | | | | | | | | |
| | Leaf Extract, Urtica Dioica | | | | | | | | |
| | (Nettle) Extract, | | | | | | | | |
| | Chamomilla Recutita | | | | | | | | |
| | (Matricaria) Flower Extract, | | | | | | | | |
| | Melissa Officinalis Leaf | | | | | | | | |
| | Extract | | | | | | | | |
| 1,,2-Decanediol | 1,,2-Decanediol | 0.20 | 1.00 | 2.00 | 0.50 | 0.20 | 0.50 | 1.00 | 2.00 |
| Dow Corning 1503 | Dimethicone, Dimethiconol | | | 2.00 | | | | | |
| Dow Corning 200 Fluid | Dimethicone | | 1.00 | | | | | | |
| Dow Corning 345 Fluid | Cyclomethicone | | | 3.00 | | | 0.50 | | |
| Dracorin 100 (Symrise) | Glyceryl Stearate, PEG- | | 6.00 | | | | | | |
| | 100 Stearate | | | | | | | | |
| Dracorin GMS (Symrise) | Glyceryl Stearate | | | | | | 0.50 | | |
| Dracorin GOC (Symrise) | Glyceryl Oleate Citrate, | | | | | | 2.00 | | 2.00 |
| | Caprylic/Capric | | | | | | | | |
| | Triglyceride | | | | | | | | |
| Drago-Beta-Glucan | Water (aqua), Butylene | | | | | | 1.50 | | |
| (Symrise) | Glycol, Glycerin, Avena | | | | | | | | |
| | Sativa (Oat) Kernel Extract | | | | | | | | |
| DragoCalm (Symrise) | Water, Glycerin, Avena | 0.50 | | | | | | | |
| | Sativa (Oat) Kernel Extract | | | | | | | | |
| Dragocide Liquid (Symrise) | Phenoxyethanol, | | | 0.80 | 0.80 | | | | 0.80 |
| | Methylparaben, | | | | | | | | |
| | Ethylparaben, | | | | | | | | |
| | Butylparaben, | | | | | | | | |
| | Propylparaben, | | | | | | | | |
| | Isobutylparaben | | | | | | | | |
| Dragoderm (Symrise) | Glycerin, Triticum Vulgare | | 2.00 | | | | | | |
| | (Wheat) Gluten, Water | | | | | | | | |
| | (Aqua) | | | | | | | | |
| Dragoxat 89 (Symrise) | Ethylhexyl Isononanoate | | | 3.00 | 3.00 | | 5.00 | | |
| EDETA BD | Disodium EDTA | | | 0.10 | | | | | |
| Emulsifier Dragil (Symrise) | Glycol Stearate SE | | | | 12.00 | | | | |
| Emulsiphos (Symrise) | Potassium Cetyl | | | 2.00 | | | | | |
| | Phosphate, Hydrogenated | | | | | | | | |
| | Palm Glycerides | | | | | | | | |
| Erythromycin | | | | 3.00 | | | | | |
| Clindamycin | | | | | | | | | |
| Ethanol pure, 96% strength | Alcohol Denat. | 15.00 | 15.00 | | | 15.00 | | | |
| Extrapone Hamamelis | Propylene Glycol, | 2.00 | | | | | | | |
| (Symrise) | Hamamelis Virginiana | | | | | | | | |
| | (Witch Hazel) | | | | | | | | |
| | Bark/Leaf/Twig Extract, | | | | | | | | |
| | Water(Aqua), Alcohol, | | | | | | | | |
| | Hamamelis Virginiana | | | | | | | | |
| | (Witch Hazel) Water, | | | | | | | | |
| | Glucose | | | | | | | | |
| Extrapone Apple (Symrise) | Propylene Glycol, Water | | | | | | | 0.20 | |
| | (Aqua), Pyrus Malus | | | | | | | | |
| | (Apple) Juice | | | | | | | | |
| Extrapone Camomile | Water(Aqua), Propylene | | | | | 1.00 | | | |
| (Symrise) | Glycol, Butylene Glycol, | | | | | | | | |
| | Chamomilla Recutita | | | | | | | | |
| | (Matricaria) Flower Extract, | | | | | | | | |
| | Glucose, Bisabolol | | | | | | | | |
| Extrapone Honey GW | Water (Aqua), Glycerin, | | | | | | | 0.20 | |
| (Symrise) | Honey (Mel) | | | | | | | | |
| Extrapone Horsetail | Water(Aqua), Propylene | 2.00 | | | | | | | |
| (Symrise) | Glycol, Glucose, | | | | | | | | |
| | Equisetum Arvense | | | | | | | | |
| | Extract | | | | | | | | |
| Extrapone Pear (Symrise) | Propylene Gylcol, Water | | | | | | | 0.20 | |
| | (Aqua), Pyrus Communis | | | | | | | | |
| | (Pear) Fruit Juice | | | | | | | | |
| Colour | Colour | | | | | 0.10 | | | |
| Farnesol (Symrise) | Farnesol | 0.20 | | | | 0.30 | 0.30 | | 0.20 |
| Frescolat ML (Symrise) | Menthyl Lactate | | | 1.00 | | | | | |
| Glycerin, 85% strength in | Glycerin | | | 3.00 | | | | | |
| water | | | | | | | | | |
| Glycerin | Glycerin | | 2.00 | | | 5.00 | | | 3.00 |
| Hydrolite-5 (1,2-pentanediol) | Pentylene Glycol | | | | | | 5.00 | 0.50 | |
| Hydroviton - 24 (Symrise) | Water (Aqua), Pentylene | | | | | | | | 1.00 |
| | Glycol, Glycerin, Sodium | | | | | | | | |
| | Lactate, Lactic Acid, | | | | | | | | |
| | Serine, Urea, Sorbitol, | | | | | | | | |
| | Sodium Chloride, Allantoin | | | | | | | | |
| Hydroviton (Symrise) | Water (Aqua), Glycerin, | 1.00 | | | | 1.00 | | | |
| | Sodium Lactate, Lactic | | | | | | | | |
| | Acid, TEA-Lactate, Serine, | | | | | | | | |
| | Urea, Sorbitol, Sodium | | | | | | | | |
| | Chloride, Allantoin | | | | | | | | |
| Isoadipate (Symrise) | Diisopropyl Adipate | | | 2.00 | | | | | |
| Isodragol (Symrise) | Triisononanoin | | | | | | 4.00 | | 2.00 |
| Potassium sorbate | Potassium Sorbate | | | | | | 0.10 | | 0.10 |
| Keltrol RD | Xanthan Gum | | | 0.20 | | | 0.15 | | |
| Cosmetic colouring BROWN | | | | 5.00 | | | | | |
| C.I. 77491, 77492, 77499, | | | | | | | | | |
| 77891 | | | | | | | | | |
| Lanette 18 | Stearyl Alcohol | | 1.5 | | | | | | |
| Lanette E | Sodium Cetearyl Sulfate | | | 0.80 | | | | | |
| Lanette O | Cetearyl Alcohol | | | 1.20 | | | 1.00 | | |
| Food colouring TITANIUM | Titanium Dioxide (CI | | | 3.00 | | | | | |
| DIOXIDE E171 | 77891) | | | | | | | | |
| Lumorol K 5019 | Disodium Laureth | | | | | | | 25.00 | |
| | Sulfosuccinate, Sodium | | | | | | | | |
| | Lauryl Sulfoacetate | | | | | | | | |
| Menthol | Menthol | 0.05 | | | | | | | |
| Lactic acid, 90% | Lacric Acid | | 2.00 | | | | | | |
| Sodium hydroxide, 10% | Sodium Hydroxide | | | | | 2.50 | 0.50 | | 0.55 |
| strength solution | | | | | | | | | |
| Neo Dragocid Liquid | Triethylene Glycol, | | | | | | | 0.50 | |
| (Symrise) | Imidazolidinyl Urea, | | | | | | | | |
| | Methylparaben, | | | | | | | | |
| | Propylparaben, | | | | | | | | |
| | Dehydroacetic Acid | | | | | | | | |
| Neo Heliopan 303 | Octocrylene | | | 1.5 | | | | | |
| Neo Heliopan 357 (Symrise) | Butyl | | | 1.20 | | | | | |
| | Methoxydibenzoylmethane | | | | | | | | |
| Neo Heliopan AP used as a | Disodium Phenyl | | | 1.36 | | | | | |
| 22% strength solution | Dibenzimidazole | | | | | | | | |
| neutralized with | Tetrasulfonate | | | | | | | | |
| triethanolamine, (Symrise) | | | | | | | | | |
| Neo Heliopan AV (Symrise) | Ethylhexyl Methoxy- | | | 4.00 | | | | | |
| | cinnamate | | | | | | | | |
| Neo-PCL ws N (Symrise) | Trideceth-9, PEG-5, | 0.50 | | | | 1.00 | | 1.50 | |
| | Ethylhexanoate, Water | | | | | | | | |
| | (Aqua) | | | | | | | | |
| Neutral oil | Caprylic/Capric | | 5.00 | | | | | | |
| | Triglyceride | | | | | | | | |
| Oxetal VD 92 | Glycereth-90 Isostearate, | | | | | | | 4.50 | |
| | Laureth-2 | | | | | | | | |
| Perfume oil | Perfume | 0.10 | 0.30 | 0.20 | 0.10 | 0.05 | 0.20 | 0.10 | 0.30 |
| PCL -Solid (Symrise) | Stearyl Heptanoate, | | | | | | 2.50 | | |
| | Stearyl Caprylate | | | | | | | | |
| PCL-Liquid 100 (Symrise) | Cetearyl Ethylhexanoate | | 2.00 | | 4.00 | | 5.00 | | 3.00 |
| Pemulen TR-2 | Acrylates/C10-30 Alkyl | | | | | | | | 0.20 |
| | Acrylate Crosspolymer | | | | | | | | |
| Pionier NP 37 N | Sodium Carbomer | | 0.10 | | | | | | |
| 1,2-Propylene glycol | Propylene Glycol | | 3.00 | | 4.00 | | | | |
| all-trans-Retinal | Retinal, all-trans | | | | | | 0.05 | | |
| all-trans-Retinoic acid | Tretinoin | | 0.05 | | | | | | |
| cis-13-Retinoic acid | Isotretinoin | | | | 0.05 | | | | |
| Salicylic acid | Salicylic Acid | | | 10.00 | | | | | |
| Solubilizer, solubility | PEG-40 Hydrogenated | 0.50 | | | | 1.50 | | 1.00 | |
| improver | Castor Oil, Trideceth-9, | | | | | | | | |
| | Propylene Glycol, | | | | | | | | |
| | Water(aqua) | | | | | | | | |
| SymCalmin (Symrise) | Butylene Glycol, Pentylene | | | | | | | | 1.00 |
| | Glycol, Hydroxyphenyl | | | | | | | | |
| | Propamidobenzoic Acid | | | | | | | | |
| SymDiol 68 (Symrise) | 1,2 Hexanediol, Caprylyl | 0.50 | | | | | | | |
| | Glycol (1:1 (m/m) | | | | | | | | |
| Tetracylcine | | | | | | | 3.00 | | |
| Tego Betain F50 | Cocamidopropyl Betaine | | | | | | | 10.00 | |
| Triethanolamine, 99% | Triethanolamine | | | 0.20 | | | | | |
| strength | | | | | | | | | |
| Water | Water (Aqua) | to 100 | to 100 | to 100 | to 100 | to 100 | to 100 | to 100 | to 100 |
| Witch Hazel Distillate | Hamamelis | | 0.10 | | | | | | |
| (Symrise) | Virginiana(Witch Hazel) | | | | | | | | |
| | Water, Water(Aqua), | | | | | | | | |
| | Alcohol | | | | | | | | |

1 = Facial water against greasy, oily skin
2 = Cleansing milk O/W against greasy, oily skin
3 = Antiacne make-up SPF 8
4 = Antiacne cream O/W
5 = Antiacne gel
6 = Antiacne lotion
7 = Washing lotion against greasy, oily skin
8 = Impregnating liquid for antiacne wipes

### Specific Embodiments

Specific embodiment one comprises the use of 1,2-decanediol for reduction of the sebum concentration of the skin.

Specific embodiment two comprises a cosmetic and/or dermatological formulation for topical application, characterized in that the formulation comprises the following constituents:
an amount of 1,2-decanediol which is sufficient to reduce the sebum concentration of the skin,
optionally one, two, three, four, five, six, seven, eight, nine, ten or more cleansing auxiliary substances,
one, two, three, four or more active compounds chosen from the group consisting of benzoyl peroxide, salicylic acid, lactic acid, azelaic acid, retinoids, antibiotics, sulfur, chlorhexidine, triclosan, farnesol, phenoxyethanol, isoflavonoids and antiandrogens, and
optionally one or more further auxiliary substances and/or additives
   with the proviso that
if the formulation contains 1 wt. % of 1,2-decanediol, then the formulation may not at the same time contain 1 wt. % lactic acid or 2 wt. % lactic acid,
if the formulation contains 0.5 wt. % 1,2-decanediol, then the formulation may not at the same time contain 0.5 wt. % salicylic acid or 1 wt. % salicylic acid or 0.5 wt. % lactic acid and 1 wt. % salicylic acid,
if the formulation contains 0.4 wt. % 1,2-decanediol, then the formulation may not at the same time contain 1 wt. % lactic acid and 0.5 wt. % salicylic acid,
if the formulation contains 0.3 wt. % 1,2-decanediol, then the formulation may not at the same time contain 2.5 wt. % lactic acid or 0.2 wt. % salicylic acid and 1 wt. % lactic acid,
if the formulation contains 0.2 wt. % 1,2-decanediol, then the formulation may not at the same time contain 2 wt. % lactic acid or 1 wt. % lactic acid or 0.5 wt. % lactic acid, and
if the formulation contains 0.1 wt. % 1,2-decanediol, then the formulation may not at the same time contain 1 wt. % lactic acid.

Specific embodiment three comprises a formulation according to specific embodiment two, characterized in that the formulation comprises one, two, three, four, five, six, seven, eight, nine, ten or more cleansing auxiliary substances chosen from the group consisting of water, ethanol, surfactants, polyethylene glycol derivatives, polyethylene glycol esters, skin moisture-regulating substance, urea, sorbitol, readily spreading cosmetic esters, skin soothing and/or antiinflammatory compounds, plant extracts and antiseborrhoeic active compounds.

Specific embodiment four comprises a formulation according to specific embodiment three, characterized in that one, two, three or more active compounds are chosen from the group of benzoyl peroxide, salicylic acid, lactic acid, azelaic acid, all-trans retinoic acid, all-trans retinal, cis-13-retinoic acid, adaptalen, clindamycin, erythromycin, tetracycline, sulfur, chlorhexidine, triclosan, farnesol, phenoxyethanol, genistein, daidzein, genistin, daizin and 5α-reductase inhibitor.

Specific embodiment five comprises a formulation according to specific embodiment three or four, characterized in that one, two, three or more active compounds are chosen from the group of benzoyl peroxide, azelaic acid, all-trans retinoic acid, all-trans retinal, cis-13-retinoic acid, adaptalen, clindamycin, erythromycin, tetracycline, sulfur, chlorhexidine, triclosan, farnesol, phenoxyethanol, genistein, daidzein, genistin, daizin and 5α-reductase inhibitor.

Specific embodiment six comprises a formulation according to any of specific embodiments three to five, characterized in that one, two, three or more active compounds are chosen from the group consisting of benzoyl peroxide, azelaic acid, all-trans-retinoic acid, all-trans-retinal, cis-13-retinoic acid, adaptalene, clindamycin, erythromycin and tetracycline.

Specific embodiment seven comprises a method for reduction of the sebum concentration of the skin, characterized in that the method comprises the following steps:
a) provision of 1,2-decanediol;
b) preparation of a formulation according to any of specific embodiments three to six and
c) application of the formulation prepared according to b) to greasy-oily and/or impure skin, the amount of 1,2-decanediol being sufficient to reduce the sebum concentration of the skin,

Specific embodiment eight comprises use of 1,2-decanediol to assist the penetration of one, two, three or more active compounds chosen from the group consisting of benzoyl peroxide, salicylic acid, lactic acid, azelaic acid, retinoids, antibiotics, sulfur, chlorhexidine, triclosan, farnesol, phenoxyethanol, isoflavonoids and antiandrogens into areas of skin with increased sebum production.

Specific embodiment nine comprises a method for assisting the penetration of one, two, three or more active compounds chosen from the group consisting of benzoyl peroxide, salicylic acid, lactic acid, azelaic acid, retinoids, antibiotics, sulfur, chlorhexidine, triclosan, farnesol, phenoxyethanol, isoflavonoids and antiandrogens in areas of skin with increased sebum production, characterized in that the method comprises the following steps:
a) provision of 1,2-decanediol,
b) provision of one, two, three or more active compounds chosen from the group consisting of benzoyl peroxide, salicylic acid, lactic acid, azelaic acid, retinoids, antibiotics, sulfur, chlorhexidine, triclosan, farnesol, phenoxyethanol, isoflavonoids and antiandrogens,
c) preparation of a cosmetic and/or dermatological formulation for topical application comprising
   an amount of 1,2-decanediol which is sufficient to reduce the sebum concentration of the skin,
   an active amount of one, two, three, four or more active compounds chosen from the group consisting of benzoyl peroxide, salicylic acid, lactic acid, azelaic acid, retinoids, antibiotics, sulfur, chlorhexidine, triclosan, farnesol, phenoxyethanol, isoflavonoids and antiandrogens
   optionally one, two, three, four, five, six, seven, eight, nine, ten or more cleansing auxiliary substances and
   optionally one or more further auxiliary substances and/or additives with the proviso that if the formulation contains a concentration of 1 wt. % lactic acid, the concentration of 1,2-decanediol may not be 0.2 wt. %, in each case based on the total formulation,
with the provisio that
if the formulation contains 1 wt. % of 1,2-decanediol, then the formulation may at the same time not contain 1 wt. % lactic acid or 2 wt. % lactic acid,
if the formulation contains 0.5 wt. % 1,2-decanediol, then the formulation may at the same time not contain 0.5 wt. % salicylic acid or 1 wt. % salicylic acid or 0,5 wt. % lactic acid and 1 wt. % salicylic acid,
if the formulation contains 0.4 wt. % 1,2-decanediol, then the formulation may at the same time not contain 1 wt. % lactic acid and 0.5 wt. % salicylic acid,
if the formulation contains 0.3 wt. % 1,2-decanediol, then the formulation may at the same time not contain 2.5 wt. % lactic acid or 0.2 wt. % salicylic acid and 1 wt. % lactic acid,
if the formulation contains 0.2 wt. % 1,2-decanediol, then the formulation may at the same time not contain 2 wt. % lactic acid or 1 wt. % lactic acid or 0.5 wt. % lactic acid, and
if the formulation contains 0.1 wt. % 1,2-decanediol, then the formulation may at the same time not contain 1 wt. % lactic acid, and
d) application of the formulation prepared according to c) to the relevant areas of the skin.

1. A method for the reduction of the sebum concentration of the skin comprising applying 1,2-decanediol.

2. A cosmetic and/or dermatological formulation for topical application, wherein the formulation comprises the following constituents:
an amount of 1,2-decanediol which is sufficient to reduce the sebum concentration of the skin,
optionally one, two, three, four, five, six, seven, eight, nine, ten or more cleansing auxiliary substances,
one, two, three, four or more active compounds chosen from the group consisting of benzoyl peroxide, salicylic acid, lactic acid, azelaic acid, retinoids, antibiotics, sulfur, chlorhexidine, triclosan, farnesol, phenoxyethanol, isoflavonoids and antiandrogens, and
optionally one or more further auxiliary substances and/or additives
with the proviso that
if the formulation contains 1 wt. % of 1,2-decanediol, then the formulation may not at the same time contain 1 wt. % lactic acid or 2 wt. % lactic acid,
if the formulation contains 0.5 wt. % 1,2-decanediol, then the formulation may not at the same time contain 0.5 wt. % salicylic acid or 1 wt. % salicylic acid or 0.5 wt. % lactic acid and 1 wt. % salicylic acid,
if the formulation contains 0.4 wt. % 1,2-decanediol, then the formulation may not at the same time contain 1 wt. % lactic acid and 0.5 wt. % salicylic acid,
if the formulation contains 0.3 wt. % 1,2-decanediol, then the formulation may not at the same time contain 2.5 wt. % lactic acid or 0.2 wt. % salicylic acid and 1 wt. % lactic acid,
if the formulation contains 0.2 wt. % 1,2-decanediol, then the formulation may not at the same time contain 2 wt. % lactic acid or 1 wt. % lactic acid or 0.5 wt. % lactic acid, and
if the formulation contains 0.1 wt. % 1,2-decanediol, then the formulation may not at the same time contain 1 wt. % lactic acid.

3. The formulation according to claim 2, wherein the formulation comprises one, two, three, four, five, six, seven, eight, nine, ten or more cleansing auxiliary substances chosen from the group consisting of water, ethanol, surfactants, polyethylene glycol derivatives, polyethylene glycol esters, skin moisture-regulating substance, urea, sorbitol, readily spreading cosmetic esters, skin soothing and/or antiinflammatory compounds, plant extracts and antiseborrhoeic active compounds.

4. The formulation according to claim 3, characterized in that one, two, three or more active compounds are chosen from the group consisting of benzoyl peroxide, salicylic acid, lactic acid, azelaic acid, all-trans retinoic acid, all-trans retinal, cis-13-retinoic acid, adaptalen, clindamycin, erythromycin, tetracycline, sulfur, chlorhexidine, triclosan, farnesol, phenoxyethanol, genistein, daidzein, genistin, daizin and 5α-reductase inhibitor.

5. The formulation according to claim 3, wherein one, two, three or more active compounds are chosen from the group consisting of benzoyl peroxide, azelaic acid, all-trans retinoic acid, all-trans retinal, cis-13-retinoic acid, adaptalen, clindamycin, erythromycin, tetracycline, sulfur, chlorhexidine, triclosan, farnesol, phenoxyethanol, genistein, daidzein, genistin, daizin and 5α-reductase inhibitor.

6. The formulation according to claim 3, wherein one, two, three or more active compounds are chosen from the group consisting of benzoyl peroxide, azelaic acid, all-trans-retinoic acid, all-trans-retinal, cis-13-retinoic acid, adaptalene, clindamycin, erythromycin and tetracycline.

7. A method for reduction of the sebum concentration of the skin, wherein the method comprises the following steps:
a) provision of 1,2-decanediol;
b) preparation of a formulation according to claim 3 and
c) application of the formulation prepared according to b) to greasy-oily and/or impure skin, the amount of 1,2-decanediol being sufficient to reduce the sebum concentration of the skin,

8. A method for assisting the penetration of one, two, three or more active compounds chosen from the group consisting of benzoyl peroxide, salicylic acid, lactic acid, azelaic acid, retinoids, antibiotics, sulfur, chlorhexidine, triclosan, farnesol, phenoxyethanol, isoflavonoids and antiandrogens into areas of skin with increased sebum production comprising applying 1,2-decanediol.

9. A method for assisting the penetration of one, two, three or more active compounds chosen from the group consisting of benzoyl peroxide, salicylic acid, lactic acid, azelaic acid, retinoids, antibiotics, sulfur, chlorhexidine, triclosan, farnesol, phenoxyethanol, isoflavonoids and antiandrogens in areas of skin with increased sebum production, the method comprising:
a. provision of 1,2-decanediol,
b. provision of one, two, three or more active compounds chosen from the group consisting of benzoyl peroxide, salicylic acid, lactic acid, azelaic acid, retinoids, antibiotics, sulfur, chlorhexidine, triclosan, farnesol, phenoxyethanol, isoflavonoids and antiandrogens,
c. preparation of a cosmetic and/or dermatological formulation for topical application comprising
an amount of 1,2-decanediol which is sufficient to reduce the sebum concentration of the skin,
an active amount of one, two, three, four or more active compounds chosen from the group consisting of benzoyl peroxide, salicylic acid, lactic acid, azelaic acid, retinoids, antibiotics, sulfur, chlorhexidine, triclosan, farnesol, phenoxyethanol, isoflavonoids and antiandrogens
optionally one, two, three, four, five, six, seven, eight, nine, ten or more cleansing auxiliary substances and
optionally one or more further auxiliary substances and/or additives with the proviso that if the formulation contains a concentration of 1 wt. % lactic acid, the concentration of 1,2-decanediol may not be 0.2 wt. %, in each case based on the total formulation,
with the provisio that
if the formulation contains 1 wt. % of 1,2-decanediol, then the formulation may at the same time not contain 1 wt. % lactic acid or 2 wt. % lactic acid,
if the formulation contains 0.5 wt. % 1,2-decanediol, then the formulation may at the same time not contain 0.5 wt. % salicylic acid or 1 wt. % salicylic acid or 0.5 wt. % lactic acid and 1 wt. % salicylic acid,
if the formulation contains 0.4 wt. % 1,2-decanediol, then the formulation may at the same time not contain 1 wt. % lactic acid and 0.5 wt. % salicylic acid,
if the formulation contains 0.3 wt. % 1,2-decanediol, then the formulation may at the same time not contain 2.5 wt. % lactic acid or 0.2 wt. % salicylic acid and 1 wt. % lactic acid,
if the formulation contains 0.2 wt. % 1,2-decanediol, then the formulation may at the same time not contain 2 wt. % lactic acid or 1 wt. % lactic acid or 0.5 wt. % lactic acid, and
if the formulation contains 0.1 wt. % 1,2-decanediol, then the formulation may at the same time not contain 1 wt. % lactic acid, and
d. application of the formulation prepared according to c) to the relevant areas of the skin.
